(19) United States
(12) Patent Application Publication
(10) Pub. No.: US 2004/0002440 A1
(43) Pub. Date: Jan. 1, 2004

(54) Novel diagnostic agents of chronic or persistent chlamydial diseases and uses thereof

(75) Inventors: Peter Timms, Queensland (AU); Sarah Anne Mathews, Queensland (AU)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Queensland University of Technology

(21) Appl. No.: 10/369,435

(22) Filed: Feb. 19, 2003

Related U.S. Application Data
(63) Continuation of application No. PCT/AU01/01021, filed on Aug. 17, 2001.

(30) Foreign Application Priority Data
Aug. 18, 2000 (AU) PQ9540

(51) Int. Cl. A61K 38/16; A61K 48/00; G01N 33/554; G01N 33/569; C12Q 1/68

(52) U.S. Cl. 514/2; 514/44; 435/6; 435/7.32

(57) ABSTRACT

The present invention discloses compositions and methods for detecting organisms of the Chlamydiaceae family, including species of Chlamydia and Chlamyclophila, in the persistent phase of their developmental cycle and for the diagnosis of chronic or persistent infections caused by such organisms. The present invention also discloses methods for screening agents that are useful inter alia for modulating a gene whose expression is altered in the persistent phase of the chiamydial developmental cycle or for modulating the level and/or functional activity of an expression product of that gene. Also disclosed are methods and compositions for the treatment and/or prophylaxis of infections, including chronic infections, caused by chamydial organisms using the aforesaid modulatory agents and optionally agents that are effective in modulating the expression of a gene associated with the lytic phase of said developmental cycle or in modulating the level and/or functional activity of an expression product of that gene. The invention also discloses methods and compositions for the treatment and/or prophylaxis of such infections using a first immunopotentiating agent that elicits the production of elements that are immuno-interactive with an antigen associated with the persistent phase of the chlamydial developmental cycle and a second immunopotentiating agent that elicits the production of elements that are immuno-interactive with an antigen associated with the lytic phase of said developmental cycle.

### CROSS-REFERENCE TO RELATED APPLICATIONS

This application is a continuation of International Patent Application No. PCTPCT/AU01/01021, filed Aug. 17, 2001, which was published in the English language on Feb. 21, 2002, under International Publication No. WO 02/14516 A1, and the disclosure of which is incorporated herein by reference.

### FIELD OF THE INVENTION

This invention relates generally to infections caused by organisms belonging to the family Chlamydiaceae. More particularly, the present invention relates to the detection of organisms of the Chlamydiaceae family, including species of Chlamydia and Chlamydophila, in the persistent phase of their developmental cycle and to the diagnosis of chronic or persistent infections caused by such organisms. The present invention also extends to the development of methods for screening agents that are useful inter alia for modulating a gene whose expression is altered in the persistent phase of said developmental cycle or for modulating the level and/or functional activity of an expression product of that gene. The invention also encompasses the treatment and/or prophylaxis of infections, including chronic infections, caused by said organisms using the aforesaid modulatory agents and optionally agents that are effective in modulating the expression of a gene associated with the lytic phase of said developmental cycle or in modulating the level and/or functional activity of an expression product of that gene. The invention also extends to the treatment and/or prophylaxis of such infections using a first immunopotentiating agent that elicits the production of elements that are immuno-interactive with an antigen associated with the persistent phase of said developmental cycle and a second immunopotentiating agent that elicits the production of elements that are immuno-interactive with an antigen associated with the lytic phase of said developmental cycle.

Bibliographic details of the publications referred to in this specification are collected at the end of the description.

### BACKGROUND OF THE INVENTION

The chlamydiae are important pathogens of humans, birds and a wide range of animals. They primarily cause disease at mucosal sites, such as the eye (trachoma), the female urogenital tract (tubal blockage and infertility in humans, abortion in animals) and the lungs (pneumonia, chronic obstructive pulmonary disease). They can also be found associated with more systemic diseases such as psittacosis and have recently been implicated in atherosclerosis. Many of the disease states caused by chlamydial infection are primarily not due to the initial lytic insult of the parasite but progress slowly over many years (eg. trachoma, tubal infertility). It has been suggested therefore that the pathogenesis of chlamydial infections is due to a host initiated hypersensitivity response to specific chlamydial antigens, resultant from chronic low grade chlamydial infection (Morrison et al., 1989). While there is some data from the late 1980s to implicate chlamydial heat shock 60 protein in this immune mediated pathogenesis (Morrison et al., 1989), this has not been well replicated and it appears likely that additional chlamydial antigens are involved.

Phylogenetically, the chlamydiae are a unique group of bacteria, characterised by a developmental cycle that involves the conversion between two distinct morphological forms. Infection begins with the attachment of the infectious elementary body (EB) to a susceptible eukaryotic cell and subsequent ingestion into a host-derived endosome. Inside this developing chlamydial inclusion, the EB differentiates into the non-infectious reticulate body (RB), which multiplies by binary fission an estimated 200-300-fold (Mathews et al., 1999). After 48-72 hours (depending on the chlamydial species and strain) the RBs reorganise back into metabolically inactive but infectious EBs, which are subsequently released upon host cell lysis. While this lytic developmental cycle is well characterised in vitro, recent evidence supports the presence of an additional, non-lytic, persistent phase of the cycle. Various authors have reported the induction of morphologically abnormal, persistent or chronic forms of chlamydiae induced by such agents as β-lactam antibiotics, D-cycloserine, IFN-δ or nutrient deprivation (Beatty et al., 1993a; Coles et al., 1993; Kramer & Gordon, 1971; Matsumoto & Manire, 1970). These persistent chlamydial forms are characterised by altered morphology (usually enlarged with aberrant shape), by being viable but non-infectious when passaged to other cells and by having altered steady-state levels of some chlamydial antigens (MOMP, OMPcB, LPS, HSP60). These persistent chlamydiae apparently are not end-stage forms that are on an irreversible path to death, as they can be reactivated by several means including removal of the IFN-δ or addition of tryptophan (Beatty et al., 1995). A recent report by Harper et al. (2000) suggests that metabolic starvation (low levels of amino acids and even glucose) results in C. trachomatis switching some of its growth to the morphologically abnormal persistent phase, and that this stress state might be a common feature of all persistent stages, induced by many different types of upstream initiators. Harper et al. (2000) go further to suggest that the normal developmental cycle for Chlamydia might only be representative of the organism growing under ideal in vitro conditions and that many in vivo conditions could result in metabolic stress causing at least some of the organisms to switch to the persistent state.

### SUMMARY OF THE INVENTION

The present inventors have surprisingly discovered that, in addition to genes encoding MOMP, OMPcB and HSP60 (ompA, ompB and hsp60) and genes involved in the biosynthesis of LPS, there are at least three other chlamydial genes, including pyk, nlpD and Cpn0585, whose steady-state expression is altered in the persistent phase of the chlamydial developmental cycle. It is believed that the expression of other chlamydial genes may also be altered in the persistent phase, particularly those genes involved in the same regulatory or biosynthetic pathways as pyk, nlpD and Cpn0585. The identification of these target genes permits the selection or rational design of agents that modulate the expression of the those genes or the level and/or functional activity of their expression products for use inter alia in the prevention and/or treatment of infections, including persistent or chronic infections, caused by an organism of the Chlamydiaceae family.

Accordingly, in one aspect of the present invention, there is provided a method for detecting an organism of the Chlamydiaceae family in the persistent phase of its developmental cycle, said method comprising detecting, relative to the lytic phase of said developmental cycle, a change in the level and/or functional activity of an expression product of a gene selected from pyk, nlpD, Cpn0585, or a gene belonging to the same regulatory or biosynthetic pathway as pyk, nlpD or Cpn0585, or a variant of said gene.

Preferably, the change is an at least 10%, more preferably at least 50%, even more preferably at least 100%, even more preferably at least 200%, even more preferably at least 400%, even more preferably at least 600% and still even more preferably at least 1000% change in said level and/or functional activity.

In another aspect, the invention features a method for diagnosis of a persistent or chronic infection in a patient, wherein said infection is caused by an organism of the Chlamydiaceae family, said method comprising detecting in a biological sample obtained from said patient, relative to the lytic phase of the developmental cycle of said organism, a change in the level and/or functional activity of an expression product of a gene selected from pyk, nlpD, Cpn0585, or a gene belonging to the same regulatory or biosynthetic pathway as pyk, nlpD or Cpn0585, or a variant of said gene.

In one embodiment, the method preferably comprises:

contacting the biological sample with an antigen-binding molecule that is immuno-interactive with a polypeptide expressed from said gene;

measuring the concentration of a complex comprising said polypeptide and the antigen binding molecule in said contacted sample; and

relating said measured complex concentration to the concentration of said polypeptide in said sample.

Preferably, the concentration of said polypeptide in said biological sample is compared to a reference level of said polypeptide corresponding to said lytic phase.

In another embodiment, the method preferably comprises:

measuring the level of a transcript expressed from said gene in said biological sample.

Preferably, the level of said transcript in said biological sample is compared to a reference level of said transcript corresponding to said lytic phase.

In yet another embodiment, the method preferably comprises:

contacting the biological sample with an antigen corresponding to at least a portion of a polypeptide encoded by said gene;

measuring the concentration of a complex comprising said antigen and an antigen-binding molecule in said contacted sample; and

relating said measured complex concentration to the concentration of antigen-binding molecule in said sample to thereby determine the amount or level of said polypeptide in said sample.

Preferably, the concentration of said antigen-binding molecule in said biological sample is compared to a reference level of said antigen-binding molecule corresponding to said lytic phase.

In still yet another embodiment, the method preferably comprises:

contacting the biological sample with an antigen corresponding to at least a portion of a polypeptide encoded by said gene;

measuring the level of antigen-specific T cell proliferation in said contacted sample to thereby determine the amount or level of said polypeptide in said sample.

Preferably, the level of said antigen-specific T cell proliferation in said biological sample is compared to a reference level of antigen-specific T cell proliferation corresponding to said lytic phase.

In yet another aspect, the invention contemplates the use of an agent in the manufacture of a medicament for treating and/or preventing persistent chlamydial infections, wherein said agent modulates the expression of a gene or the level and/or functional activity of an expression product of said gene, wherein said gene is selected from pyk, nlpD, Cpn0585, or a gene belonging to the same regulatory or biosynthetic pathway as pyk, nlpD or Cpn0585, or a variant of said gene, and has been identified by a screening assay comprising:

contacting a preparation comprising a polypeptide encoded by said gene, or biologically active fragment of said polypeptide, or variant or derivative of these, or a genetic sequence that modulates the expression of said gene, with said agent; and

detecting a change in the level and/or functional activity of said polypeptide or biologically active fragment thereof, or variant or derivative, or of a product expressed from said genetic sequence.

In still yet another aspect, the invention provides the use of an agent as broadly described above in the manufacture of a medicament for treatment and/or prophylaxis of chronic infection caused by an organism of the Chlamydiaceae family, wherein said agent is formulated with a pharmaceutically acceptable carrier and/or diluent.

In still another aspect, the invention provides a method of modulating the expression of a gene or the level and/or functional activity of an expression product of said gene, wherein said gene is selected from pyk, nlpd, Cpn0585, or a gene belonging to the same regulatory or biosynthetic pathway as pyk, nlpD or Cpn0585, or a variant of said gene, said method comprising contacting a cell containing said gene with an agent for a time and under conditions sufficient to modulate the expression of said gene or the level and/or functional activity of said expression product.

In a further aspect, the invention contemplates a method for the treatment and/or prophylaxis of a chronic infection caused by an organism of the Chlamydiaceae family in a patient, said method comprising administering to said patient an effective amount of an agent that modulates the expression of a gene or the level and/or functional activity of an expression product of said gene, wherein said gene is selected from pyk, nlpD, Cpn0585, or a gene belonging to the same regulatory or biosynthetic pathway as pyk, nlpD or Cpn0585, or a variant of said gene for a time and under conditions sufficient to treat and/or prevent said infection.

Still another aspect of the present invention encompasses a method for treatment and/or prophylaxis of a lytic or chronic infection caused by an organism of the Chlamydiaceae family in a patient, said method comprising sequentially or simultaneously administering to said patient effective amounts of a first agent and a second agent for a time and under conditions sufficient to treat and/or prevent said infection, wherein said first agent modulates the expression of a first gene that is expressed at a higher level in the persistent phase of the developmental cycle of said organism than in the lytic phase of said developmental cycle or modulates the level and/or functional activity of an expression product of said first gene, and wherein said second agent modulates the expression of a second gene that is expressed at a higher level in said lytic phase than in said persistent phase or modulates the level and/or functional activity of an expression product of said second gene.

In a preferred embodiment, the first gene is selected from pyk, nlpD or Cpn0585, or a gene belonging to the same regulatory or biosynthetic pathway as pyk, nlpD or Cpn0585, or a variant of these.

In another embodiment, the second agent is an antibiotic effective in treating and/or preventing said lytic infection.

In another embodiment, the second agent is immuno-interactive with an antigen expressed in the lytic phase of said developmental cycle.

Still yet another aspect of the present invention features a method for treatment and/or prophylaxis of a lytic or chronic infection caused by an organism of the Chlamydiaceae family in a patient, said method comprising sequentially or simultaneously administering to said patient an effective amount of a first agent that modulates the expression of a first gene that is expressed in the persistent phase of the developmental cycle of said organism at a higher level than in the lytic phase of said developmental cycle, or that modulates the level and/or functional activity of an expression product of said first gene, for a time and under conditions sufficient to cause said organism to enter said lytic phase, together with an effective amount of a second agent that modulates the expression of a second gene that is expressed at higher level in said lytic phase than in said persistent phase or that modulates the level and/or functional activity of an expression product of said second gene, for a time and under conditions sufficient to kill, attenuate or otherwise inactivate said organism.

Still a further aspect of the present invention envisions a method for treatment and/or prophylaxis of a lytic or chronic infection caused by an organism of the Chlamydiaceae family in a patient, said method comprising sequentially or simultaneously administering to said patient effective amounts of a first immunopotentiating agent and a second immunopotentiating agent for a time and under conditions sufficient to treat and/or prevent said infection, said first immunopotentiating agent being selected from a first proteinaceous molecule comprising at least a portion of a polypeptide, or variant or derivative thereof, that is expressed at a higher level in the persistent phase of the developmental cycle of said organism than in the lytic phase of said developmental cycle, or a polynucleotide from which said first proteinaceous molecule is expressible, said second immunopotentiating agent being selected from a second proteinaceous molecule comprising at least a portion of a polypeptide, or a variant or derivative thereof, that is expressed at a higher level in said lytic phase than in said persistent phase, or a polynucleotide from which said second proteinaceous molecule is expressible.

In yet another aspect of the present invention there is provided a method for treatment and/or prophylaxis of a lytic or chronic infection caused by an organism of the Chlamydiaceae family in a patient, said method comprising sequentially or simultaneously administering to said patient effective amounts of a first antigen that is expressed at a higher level in the persistent phase of the developmental cycle of said organism than in the lytic phase of said developmental cycle, and a second that is expressed a higher level in said lytic phase than in said persistent phase.

In another aspect, the invention provides the use of an antigen that is expressed a higher level in the persistent phase of the developmental cycle of an organism of the Chlamydiaceae family than in the lytic phase of said developmental cycle in the manufacture of a medicament for treating or preventing chronic chlamydial infection, wherein said antigen is formulated with a pharmaceutically acceptable carrier and/or diluent.

Suitably, said composition further comprises an adjuvant. Preferably, the adjuvant is a mucosal adjuvant.

Suitably, the composition further comprises at least one additional antigen. The additional antigen(s) may be selected from other antigens that are expressed at a higher level in said persistent phase than in said lytic phase or from of antigens that are expressed at a higher level in said lytic phase than in said persistent phase.

The antigen may be in the form of a full-length polypeptide, which is expressed by said organism, or a biologically active fragment thereof, or variant or derivative of these.

In still yet another aspect, the invention envisions an immunopotentiating composition for use in treating or preventing a chronic infection caused by an organism of the Chlamydiaceae family, comprising a first antigen that is expressed at a higher level in the persistent phase of the developmental cycle of said organism than in the lytic phase of said developmental cycle and a second antigen that is expressed at a higher level in said lytic phase than in said persistent phase, together with a pharmaceutically acceptable carrier and/or diluent.

In another aspect, the invention extends to the use of at least one antigen that is expressed at a higher level in the persistent phase of the developmental cycle of an organism of the Chlamydiaceae family than in the lytic phase of said developmental cycle in the manufacture of a medicament for treating and/or preventing chronic chlamydial infection in a patient.

In yet another aspect, the invention contemplates the use of at least one antigen that is expressed at a higher level in the persistent phase of the developmental cycle of an organism of the Chlamydiaceae family than in the lytic phase of said developmental cycle, together with at least one antigen that is expressed at a higher level in said lytic phase than in said persistent phase in the manufacture of a medicament for treating and/or preventing chlamydial infection in a patient.

### BRIEF DESCRIPTION OF THE FIGURES

FIG. 1: Transmission electron micrographs of C. pneumoniae IOL-207 infected HEp2 cell cultures either (a) untreated (EB, elementary body; RB, reticulate body; IB intermediate body); or (b) treated with IFN-δ (→) indicates pleomorphic RBs (AB, aberrant body) exhibiting abnormal budding/branching.

FIG. 2: RT-PCR analysis of gene transcript levels in normal (N) and IFN-δ-treated (IFN-gamma) C. pneumoniae cell cultures. Panel A shows an ethidium bromide stained gel for the highly transcribed genes 16SrRNA (equal between N and IFN treatments) versus ompA (upregulated in IFN treated cultures). Panel B shows an autoradiograph for analysis of the lower level gene transcripts from Cpn0585 (upregulated in normal compared to IFN-δ-treated cultures) again using 16SrRNA as an internal control.

FIG. 3: RT-PCR analysis of gene transcript levels in normal (N) and IFN-δ-treated (IFN-gamma) C. pneumoniae cell cultures for all 14 genes analysed. Genes with unaltered levels of transcription are indicated with an asterisk (*) while those that are upregulated in IFN-δ-treated cultures (persistent) are indicated by underlining.

### BRIEF DESCRIPTION OF THE SEQUENCES: SUMMARY TABLE

TABLE A

| SEQUENCE ID | | |
|---|---|---|
| NUMBER | SEQUENCE | LENGTH |
| SEQ ID NO:1 | Polynucleotide corresponding to the Cpn0585 gene of C. | 2019 nts |
| | pneumoniae | |
| SEQ ID NO:2 | Polypeptide sequence encoded by the polynucleotide | 672 aa |
| | depicted in SEQ ID NO:1 | |
| SEQ ID NO:3 | Polynucleotide sequence corresponding to the nlpD gene | 738 nts |
| | of C. pneumoniae | |
| SEQ ID NO:4 | Polypeptide sequence encoded by the polynucleotide | 245 aa |
| | sequence of SEQ ID NO:3 | |
| SEQ ID NO:5 | Polynucleotide sequence corresponding to the ompA gene | 1185 nts |
| | of C. pneumoniae | |
| SEQ ID NO:6 | Polypeptide sequence encoded by the polynucleotide | 394 aa |
| | sequence of SEQ ID NO:5 | |
| SEQ ID NO:7 | Polynucleotide sequence corresponding to the ompB gene | 1047 nts |
| | of C. pneumoniae | |
| SEQ ID NO:8 | Polypeptide sequence encoded by the polynucleotide | 348 aa |
| | sequence of SEQ ID NO:7 | |
| SEQ ID NO:9 | Polynucleotide sequence corresponding to the pyk gene of | 1461 nts |
| | C. pneumoniae | |
| SEQ ID NO:10 | Polypeptide sequence encoded by the polynucleotide | 486 aa |
| | sequence of SEQ ID NO:9 | |
| SEQ ID NO:11 | Polynucleotide sequence corresponding to the | 1665 nts |
| | omcB/ompB gene of C. trachomatis D | |
| SEQ ID NO:12 | Polypeptide sequence encoded by the polynucleotide | 554 aa |
| | sequence of SEQ ID NO:11 | |
| SEQ ID NO:13 | Polynucleotide sequence corresponding to the ompA gene | 1203 nts |
| | of C. trachomatis D | |
| SEQ ID NO:14 | Polypeptide sequence encoded by the polynucleotide | 400 aa |
| | sequence of SEQ ID NO:13 | |
| SEQ ID NO:15 | Polynucleotide sequence corresponding to the nlpD gene | 768 nts |
| | of C. trachomatis D | |
| SEQ ID NO:16 | Polypeptide sequence encoded by the polynucleotide | 255 aa |
| | sequence of SEQ ID NO:15 | |
| SEQ ID NO:17 | Polynucleotide sequence corresponding to the pyk gene of | 1494 nts |
| | C. trachomatis D | |
| SEQ ID NO:18 | Polypeptide sequence encoded by the polynucleotide | 497 aa |
| | sequence of SEQ ID NO:17 | |
| SEQ ID NO:19 | Polynucleotide sequence corresponding to the ompA gene | 1161 nts |
| | of C. trachomatis MoPn | |
| SEQ ID NO:20 | Polypeptide sequence encoded by the polynucleotide | 387 aa |
| | sequence of SEQ ID NO:19 | |
| SEQ ID NO:21 | Polynucleotide sequence corresponding to the pyk gene of | 1443 nts |
| | C. trachomatis MoPn | |
| SEQ ID NO:22 | Polypeptide sequence encoded by the polynucleotide | 481 aa |
| | sequence of SEQ ID NO:21 | |
| SEQ ID NO:23 | Polynucleotide sequence corresponding to the | 1662 nts |
| | omcB/ompB gene of C. trachomatis MoPn | |
| SEQ ID NO:24 | Polypeptide sequence encoded by the polynucleotide | 554 aa |
| | sequence of SEQ ID NO:23 | |
| SEQ ID NO:25 | Polynucleotide sequence corresponding to the nlpD gene | 729 nts |
| | of C. trachomatis MoPn | |
| SEQ ID NO:26 | Polypeptide sequence encoded by the polynucleotide | 243 aa |
| | sequence of SEQ ID NO:25 | |
| SEQ ID NO:27 | Polynucleotide sequence corresponding to the ompA gene | 1167 nts |
| | of C. pneunoniae AR039. | |
| SEQ ID NO:28 | Polypeptide sequence encoded by the polynucleotide | 389 aa |
| | sequence of SEQ ID NO:27 | |
| SEQ ID NO:29 | Polynucleotide sequence corresponding to the | 1668 nts |
| | omcB/ompB gene of C. pneumoniae AR039 | |
| SEQ ID NO:30 | Polypeptide sequence encoded by the polynucleotide | 556 aa |
| | sequence of SEQ ID NO:29 | |
| SEQ ID NO:31 | Polynucleotide sequence corresponding to the pyk gene of | 1452 nts |
| | C. pneumoniae AR039 | |
| SEQ ID NO:32 | Polypeptide sequence encoded by the polynucleotide | 484 aa |
| | sequence of SEQ ID NO:31 | |
| SEQ ID NO:33 | Polynucleotide sequence corresponding to the Cpn0585 | 1953 nts |
| | gene of C. pneumoniae AR039 | |
| SEQ ID NO:34 | Polypeptide sequence encoded by the polynucleotide | 651 aa |
| | sequence of SEQ ID NO:33 | |
| SEQ ID NO:35 | Polynucleotide sequence corresponding to a nlpD | 699 nts |
| | homologue of C. pneumoniae AR039 | |
| SEQ ID NO:36 | Polypeptide sequence encoded by the polynucleotide | 233 aa |
| | sequence of SEQ ID NO:35 | |
| SEQ ID NO:37 | Sequence of first mentioned peptide in Example 3 | 18 aa |
| SEQ ID NO:38 | Sequence of second mentioned peptide in Example 3 | 16 aa |
| SEQ ID NO:39 | Sequence of third mentioned peptide in Example 3 | 16 aa |
| SEQ ID NO:40 | Sequence of fourth mentioned peptide in Example 3 | 16 aa |
| SEQ ID NO:41 | Ct16S-F2 primer, Table 1 | 20 nts |
| SEQ ID NO:42 | Ct16S-R primer, Table 1 | 20 nts |
| SEQ ID NO:43 | CpnompA-F primer, Table 1 | 20 nts |
| SEQ ID NO:44 | CpnompA-R primer, Table 1 | 20 nts |
| SEQ ID NO:45 | CpnompB-F primer, Table 1 | 20 nts |
| SEQ ID NO:46 | CpnompB-R primer, Table 1 | 20 nts |
| SEQ ID NO:47 | CpnomcB-F primer, Table 1 | 20 nts |
| SEQ ID NO:48 | CpnomcB-R primer, Table 1 | 20 nts |
| SEQ ID NO:49 | Cpn76kDa-F primer, Table 1 | 30 nts |
| SEQ ID NO:50 | Cpn76kDa-R primer, Table 1 | 28 nts |
| SEQ ID NO:51 | Cpnpmp1-F primer, Table 1 | 20 nts |
| SEQ ID NO:52 | Cpnpmp1-R primer, Table 1 | 20 nts |
| SEQ ID NO:53 | CpngltX-F primer, Table 1 | 20 nts |
| SEQ ID NO:54 | CpngltX-R primer, Table 1 | 20 nts |
| SEQ ID NO:55 | Cpnhsp60B-F primer, Table 1 | 20 nts |
| SEQ ID NO:56 | Cpnhsp60AI-R primer, Table 1 | 20 nts |
| SEQ ID NO:57 | CpnyaeT-F primer, Table 1 | 20 nts |
| SEQ ID NO:58 | CpnyaeT-R primer, Table 1 | 20 nts |
| SEQ ID NO:59 | Cpnpyk-F primer, Table 1 | 20 nts |
| SEQ ID NO:60 | Cpnpyk-R primer, Table 1 | 20 nts |
| SEQ ID NO:61 | CpnnlpD-F primer, Table 1 | 20 nts |
| SEQ ID NO:62 | CpnnlpD-R primer, Table 1 | 20 nts |
| SEQ ID NO:63 | Cpn0585-F primer, Table 1 | 20 nts |
| SEQ ID NO:64 | Cpn0585-R primer, Table 1 | 20 nts |
| SEQ ID NO:65 | Cpn1046-F primer, Table 1 | 20 nts |
| SEQ ID NO:66 | Cpn1046-R primer, Table 1 | 20 nts |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “agent” is meant a naturally occurring or synthetically produced molecule which interacts either directly or indirectly with a target member, the level and/or functional activity of which are to be modulated.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

By “associated with the persistent phase” or “associated with the lytic phase” and the like is meant a molecule that is expressed at a higher level and/or functional activity in one of said phases relative to the other of said phases. Suitably, a selected molecule in a particular phase of the chlamydial developmental cycle is associated with that phase if it's level and/or functional activity is at least 110%, more preferably at least 150%, even more preferably at least 200%, even more preferably at least 300%, even more preferably at least 500% and still even more preferably at least 1000% of the level and/or functional activity of that molecule in the other phase of said developmental cycle.

As used herein, the term “binds specijically” and the like refers to antigen-binding molecules that bind the polypeptide or polypeptide fragments of the invention but do not significantly bind to homologous prior art polypeptides.

By “biologically active fragment” is meant a fragment of a full-length parent polypeptide which fragment retains the activity of the parent polypeptide. As used herein, the term “biologically active fragment” includes deletion mutants and small peptides, for example of at least 10, preferably at least 20 and more preferably at least 30 contiguous amino acids, which comprise the above activities. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may be extracted, untreated, treated, diluted or concentrated from an animal. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. Preferably, the biological sample is selected from a mucosal swab, a sputum sample, a throat swab, an aspirate, a nasopharyngeal aspirate, bronchio-alveolar lavage fluids and blood, including whole blood, serum and plasma.

The term “chlamydial” as used herein refers to an element, function, activity, property or feature associated with an organism belonging to the family Chlamydiaceae.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules.

By “effective amount”, in the context of treating or preventing an infection, preferably a chronic chlamydial infection, is meant the administration of that amount of active to an individual, either in a single dose or as part of a series, that is effective for treatment or prophylaxis of that infection. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “function” refers to a biological, enzymatic, or therapeutic function.

“Homology” refers to the percentage number of amino acids that are identical or constitute conservative substitutions as defined in Table A infra. Homology may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP. Variant peptides or polypeptides, isolated from a species of a genus belonging to the family Chlamydiaceae, may comprise conservative amino acid substitutions. Exemplary conservative substitutions in a polypeptide or polypeptide fragment according to the invention are recited the following table:

TABLE B

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “immuno-interactive fragment” is meant a fragment of a parent polypeptide, which fragment elicits an immune response, including the production of elements that specifically bind to said polypeptide, or variant or derivative thereof. As used herein, the term “immuno-interactive fragment” includes deletion mutants and small peptides, for example of at least six, preferably at least 8 and more preferably at least 20 contiguous amino acids, which comprise antigenic determinants or epitopes. Several such fragments may be joined together.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked” is meant that transcriptional and translational regulatory nucleic acids are positioned relative to a polypeptide-encoding polynucleotide in such a manner that the polynucleotide is transcribed and the polypeptide is translated.

The term “patient” refers to patients of human or other animals including birds, and includes any individual it is desired to examine or treat using the methods of the invention. However, it will be understood that “patient” does not imply that symptoms are present. Suitable mammals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes).

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring variants such as allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides whose sequence is distinguished from a reference polypeptide by substitution, deletion or addition of at least one amino acid. It is well understood in the art that some amino acids may be changed to others with broadly similar properties without changing the nature of the activity of the polypeptide (conservative substitutions) as described above in Table B.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Probes can be labelled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 50 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation. The higher the stringency, the higher will be the degree of complementarity between immobilised nucleotide sequences and the labelled polynucleotide sequence.

“Stringent conditions” refers to temperature and ionic conditions under which only nucleotide sequences having a high frequency of complementary bases will hybridise. The stringency required is nucleotide sequence dependent and depends upon the various components present during hybridisation. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridises to a complementary probe.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

As used herein, underscoring or italicising the name of a gene shall indicate the gene, in contrast to its protein product, which is indicated by the name of the gene in the absence of any underscoring or italicising. For example, “nlpD” shall mean the nlpD gene, whereas “NlpD” shall indicate the protein product of the “nlpD” gene.

2. Method of Modulating the Level and/or Functional Activity of a Target Molecule Whose Level and/or Functional activity is Altered in the Persistent Phase of the Chlamydial Developmental Cycle

The present invention is predicated in part on the determination that various genes of organisms belonging the Chlamydiaceae family are differentially expressed between the lytic phase and the persistent phase of their developmental cycle. In particular, the present inventors have discovered that several genes are modulated (e.g., upregulated) in the persistent phase, relative to the lytic phase, of the chlamydial developmental cycle. Not wishing to be bound by any one particular theory or mode of operation, the present inventors consider that alterations in the level and/or functional activity of the expression products (e.g., transcripts and polypeptides) of those genes may be implicated in the pathophysiology of persistent or chronic infections caused by chlamydial organisms. Accordingly, it is believed that by modulating the expression of those genes or the level and/or functional activity of their expression products, the chlamydial organisms will switch from the persistent phase to the lytic phase, thereby promoting accessibility to the immune system or to other therapeutic strategies.

The invention, therefore, provides a method of modulating the expression of a gene or the level and/or functional activity of an expression product of said gene, wherein said gene is selected from pyk, nlpD or Cpn0585, or a gene belonging to the same regulatory or biosynthetic pathway as pyk, nlpD or Cpn0585, or a variant of said gene. The method comprises contacting a cell containing said gene with an agent for a time and under conditions sufficient to modulate the expression of said gene or the level and/or functional activity of said expression product. Preferably, the change is an at least 10%, more preferably at least 50%, even more preferably at least 100%, even more preferably at least 200%, even more preferably at least 400%, even more preferably at least 600% and still even more preferably at least 1000% change in said level and/or functional activity.

Any cell is contemplated by the present invention, which contains a polynucleotide from which a transcript or polypeptide of said gene can be expressed. The cell may be selected from a prokaryotic cell including, but not restricted to, a bacterial cell or a eukaryotic cell such as a yeast cell, an insect cell or an animal cell. The cell is preferably an epithelial cell or cell line that is infected or infectable with an organism of the Chlamydiaceae family. The family Chlamydiaceae has recently been redefined by Everett et al. (1999, International Journal of Systematic Bacteriology 49(Part 2): 415-440) and, for all intended purposes, it shall be understood that the species of the invention may be an organism already known to belong to this family or that is identified and characterised in the future to belong to this family. Suitably, the organism belongs to a genus selected from Chlamydia and Chlamydophila. For example, the organism may be selected from a species including, but not limited to, Chlamydia trachomatis, Chlamydia muridarum, Chlamydia suis, Chlamydophila pecorum, Chlamydophila pneumoniae, Chlamydophila psittaci, Chlamydophila abortus, Chlamydophila caviae, and Chlamydophila felis. Preferably, the species is Chlamydophila pneumoniae.

The cell may be obtained from the epithelium of the genital tract, respiratory tract or conjunctiva or from arthritic joints. Alternatively, the cell may be a circulating macrophage, which is suitably infected with a chlamydial species such as Chlamydophila pneumoniae, or it may be associated with atherosclerotic plaque tissue from any suitable site (e.g., heart, arteries, veins, brain and periphery) or multiple sclerosis brain tissue.

Suitably, the cell contains a vector comprising a polynucleotide encoding an expression product of said gene, or a biologically active fragment of said expression product, or a variant or derivative of these, and operably linked to a regulatory nucleic acid molecule, which preferably includes a natural transcriptional element (e.g., promoter) relating to said gene. In another embodiment, the cell contains a vector comprising the regulatory polynucleotide relating to said gene operably connected to a polynucleotide encoding a reporter molecule of choice. Alternatively, the cell can be infected with a species of a genus belonging to the family Chlamydiaceae, which naturally or artificially includes said genes.

In accordance with the present invention, the agent modulates the expression of a gene or the level and/or functional activity of an expression product of said gene, wherein said gene is selected from pyk, nlpD or Cpn0585, or a gene belonging to the same regulatory or biosynthetic pathway as pyk, nlpD or Cpn0585, or a variant of said gene.

The pyk gene encodes pyruvate kinase involved in glycolysis. Exemplary pyruvate kinase (Pyk) polypeptides or variants include, but are not restricted to, CP0677 of C. pneumoniae AR39, CPn0097 of C. pneumoniae CWL029, Pyk of C. pnieumoniae J138, CT332 of C. trachomatis serovar D and TC0609 of C. trachomatis MoPn.

Other glycolytic pathway related genes include, but are not limited to, mrsA (encoding phosphomannomutase), pfkA_{—}1 (encoding fructose 6-phosphate 1-phosphotransferase), pfkA_{—}2 (encoding fructose 6-phosphate 1-phosphotransferase), dhnA (predicted to encode 1,6-fructose biphosphate aldolase), gapA (encoding glyceraldehyde-3-phosphate dehydrogenase), pgk (encoding phosphoglycerate kinase), eno (encoding enolase), pgmA (encoding phosphoglycerate mutase), pgm (encoding phosphoglucomutase), pgi (encoding glucose-6-phosphate isomerase), and tpiS (encoding triosephosphate isomerase).

The Cpn0585 gene encodes a polypeptide with similarity to C. psittaci IncA_{—}2, otherwise known as inclusion membrane protein A, which is required for fusion of chlamydial inclusions. Exemplary polypeptides or variants of this type include, but are not restricted to, CP0163 of C. pneumoniae AR39, CPn0585, of C. pneumoniae CWL029 and CPj0585 of C. pneumoniae J138.

Other inclusion membrane related genes linked by pathway to Cpn0585 include, but are not limited to, Cpn0186, incB (encoding inclusion membrane protein B) and incC (encoding inclusion membrane protein C). Representative examples of IncA polypeptides or variants include CP0581 of C. pneumoniae AR39, CPn0186 of C. pneumoniae CWL029, CPn0186 of C. pneumoniae J138, TC0396 of C. trachomatis MoPn and CT119 of C. trachomatis serovar D. Representative examples of IncB polypeptides or variants include CP0467 of C. pneumoniae AR39, CPn0291 of C. pneumoniae CWL029, IncB of C. pneumoniae J138, CT232, C. trachomatis serovar D and TC0503 of C. trachomatis MoPn. Representative examples of IncC polypeptides or variants include CP0466 of C. pneumoniae AR39, CPn0292 of C. pneumoniae CWL029 and IncC of C. pneumoniae J138.

The nlpD gene encodes a polypeptide with significant similarity to the Listeria welshimeri p60 invasin associated protein and to CPn0902 nlpD muraminidase (invasin repeat family). Exemplary polypeptides or variants of this type include, but are not restricted to, CP0964 of C. pneumoniae AR39, CPn0902 of C. pneumoniae CWL029, NlpD of C. pneumoniae J138, CT759 of C. trachomatis serovar D and TC0140 of C. trachomatis MoPn.

Cell envelope- or peptidoglycan synthesis-related genes linked by pathway to nlpD include, but are not limited to, amiA (encoding N-acetylmuramoyl-L-alanine amidase, murE (encoding UDP-N-acetylmuramoylalanyl DAP ligase), pbp3 (encoding transglycolase/transpeptidase), yabC (encoding Pbp2B family methyltransferase), murA (encoding UDP-N-acetylglucosamine 1-carboxyvinyltransferase), dacF (encoding D-alanyl-D-alanine carboxypeptidase), pbpB (encoding PbpP2 transglycolase/transpeptidase), amiB (encoding N-acetylmuramoyl-L-Ala amidase), glmU (encoding UDP-N-acetylglucosamine pyrophosphorylase), murF (encoding UDP-N-acetylmuramoyl DAP ligase), mraY (encoding muramoyl-pentapeptide transferase), murD (encoding UDP-N-acetylmuramoylalanine-glutamate ligase), murG (encoding peptidoglycan transferase), murC and ddlA (encoding UDP-N-acetylmuramate-alanine ligase and D-Ala-D-Ala ligase, respectively), glmS (encoding glucosamine-fructose-6-P aminotransferase) and murB (encoding UDP-N-acetylenolpyruvoylglucosamine reductase).

Non-limiting examples of polynucleotide sequences corresponding to the pyk, nlpD, Cpn0585, ompA and ompB genes of various chlamydial species are set forth in SEQ ID NO: 9, 17, 21 and 31, SEQ ID NO: 3, 15, 25 and 35, SEQ ID NO: 1 and 33, SEQ ID NO: 5, 13, 19 and 27 and SEQ ID NO: 7, 11, 23 and 29, respectively.

Other genes involved in the same regulatory or biosynthetic pathways as those mentioned above may be identified by analysis of target polypeptide—binding partner interactions. Such identification can be carried out, for example, using the yeast Two-Hybrid™ system, which takes advantage of transcriptional factors that are composed of two physically separable, functional domains (Chen et al., 1991, Proc Natl Acad Sci U S A 88(21): 9578-9582; Phizicky and Fields, 1994, Microbiol. Rev. 59(1): 94-123). The most commonly used transcriptional factor used in this system is the yeast GAL4 transcriptional activator consisting of a DNA binding domain and a transcriptional activation domain. Vectors are constructed to encode two hybrid proteins. One hybrid consists of the DNA-binding domain of the yeast transcriptional activator protein GAL4 fused to a known protein; the other hybrid consists of the GAL4 activation domain fused to protein sequences encoded by an expression library. Thus, two different cloning vectors are used to generate separate fusions of the GAL4 domains to genes encoding potential binding proteins. The fusion proteins are co-expressed, targeted to the nucleus and, if interactions occur, activation of a reporter gene (e.g., lacZ) produces a detectable phenotype. In the present case, for example, S. cerevisiae is transformed with a vector expressing a fusion protein comprising a target molecule of the invention together with the GALA binding domain. The S. cerevisiae is co-transformed with a second vector expressing a second fusion protein comprising another protein encoded by a chlamydial expression library together with the GAL4 activation domain. The second vector is suitably constructed using a chlamydial expression library. Such expression libraries may be formed by any suitable technique known to persons of skill in the art. Methods for producing chlamydial expression libraries are described, for example, by Neurath et al. (1999, Biologicals 27(1): 11-21), Bannantine et al. (1998, Molecular Microbiology 28(5): 1017-1026) Knudsen et al. (1999, Infection & Immunity 67(1): 375-383), Pham et al. (1998, Journal of Clinical Microbiology 36(7): 1919-1922) and Zhang et al. (1997, Archives of Biochemistry & Biophysics 344(1): 43-52). If lacZ is used as the reporter gene, co-expression of the fusion proteins will produce a blue colour if there is interaction between the two co-expressed fusion proteins. Chlamydial proteins thus identified by this system could then be tested to determine whether their levels and/or functional activities are altered in the persistent phase of the chlamydial developmental cycle.

The present inventors have found that pyk, nlpD and Cpn0585 are expressed at significantly elevated levels in the persistent state and are, therefore, ideal targets for agents that will abrogate or otherwise reduce the level and/or functional activity of their encoded protein products in the chronically infected host cells, to thereby kill or otherwise inactivate or attenuate these persistent chlamydial forms or to cause them to revert or enter the lytic phase of the chlamydial developmental cycle. It is possible that such agents would most likely be chlamydial-specific and could, therefore, be used for more extended periods than conventional antibiotics, which might prove more efficacious in eliminating these chronic infections. Accordingly, in one embodiment, the agent reduces the expression of said gene or the level and/or functional activity of said expression product. In a preferred embodiment of this type, the agent reduces, abrogates or otherwise impairs the expression of pyk, nlpD or Cpn0585, or the level and/or functional activity of an expression product of these genes.

Agents that may be used to reduce or abrogate gene expression include, but are not restricted to, oligoribonucleotide sequences, including anti-sense RNA and DNA molecules and ribozymes, that function to inhibit the translation of mRNA relating to one or more of said genes. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a target gene, are preferred.

Ribozymes are enzymatic RNA molecules capable of catalysing the specific cleavage of RNA. The mechanism of ribozyme action involves sequence specific hybridisation of the ribozyme molecule to complementary target RNA, followed by a endonucleolytic cleavage. Within the scope of the invention are engineered hammerhead motif ribozyme molecules that specifically and efficiently catalyse endonucleolytic cleavage of RNA sequences relating to said target molecules. Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridisation with complementary oligonucleotides, using ribonuclease protection assays.

Both anti-sense RNA and DNA molecules and ribozymes may be prepared by any method known in the art for the synthesis of RNA molecules. These include techniques for chemically synthesising oligodeoxyribonucleotides well known in the art such as for example solid phase phosphoramidite chemical synthesis. Alternatively, RNA molecules may be generated by in vitro or in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesise antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

The present invention also contemplates use in the above method of gene or expression product inhibitors identified by a method described for example in Section 3, infra.

In another embodiment, the agent increases, enhances or otherwise elevates the expression of said gene or the level and/or functional activity of said expression product. In a preferred embodiment of this type, the agent increases, enhances or otherwise elevates the expression of a gene (e.g., a negative regulator) or the level and/or functional activity of an expression product of said gene, which reduces, abrogates or otherwise impairs the expression of pyk, nlpD or Cpn0585, or the level and/or functional activity of an expression product of pyk, nlpD or Cpn0585. Any suitable inducers or stabilising/activating agents may be used in this regard and these can be identified or produced by methods for example disclosed in Section 3 infra. Alternatively, such an agent may comprise a polynucleotide, which encodes a negative regulator of one or more of pyk, nlpD or Cpn0585, or a polypeptide, which reduces, abrogates or otherwise impairs the level and/or functional activity of one or more expression products of these genes.

The modulatory agent of the invention will suitably promote or affect the switching of the species from the persistent phase to the lytic phase or will promote death of the species in the persistent phase. Any suitable assay of the lytic phase is contemplated by the present invention. For example, viable elementary bodies (EBs) may be detected in a cell or tissue sample by culture, which is indicative of a lytic infection. Alternatively, morphology based assays may be employed using, for example, transmission electron microscopy (TEM), direct immunofluorescence antibody staining (DFA) or phase contrast microscopy as is known in the art. EBs are easily distinguished because they are small (200 nm) and spherical, they have an electron dense nucleoid and uniform outer membrane structure by TEM and are substantially spherical with intensely stained outer membrane by DFA. RBs range in size from 500-800 nm and are uniformly spherical with low-density to high-density nucleoid with structured outer membrane by TEM and strong (but not as strong as EB) staining by DFA. Inclusions stained by DFA show high levels of fluorescence in a spherical area where the individual chlamydial particles can be distinguished. In contrast, particles involved in “chronic” infections are typically larger than RBs (800-1500 nm) and usually do no stain as well by DFA. Using TEM, chronic infection related particles have an unstructured outer membrane and the nucleoid appears dispersed compared to the EB and RB. Antigen-binding molecules, preferably monoclonal antibodies that are immuno-interactive with the genus specific-LPS or species specific-MOMP may be employed for DFA. Alternatively, a nucleic acid based assay, preferably reverse transcriptase polymerase chain reaction (RT-PCR), may be used to quantify the level of expression in a biological sample of a gene selected from pyk, nlpD, Cpn0585 or a gene belonging to the same regulatory or biosynthetic pathway as pyk, nlpD or Cpn0585, or a variant of said gene.

3. Identification of Target Molecule Modulators

The invention also features a method of screening for an agent that modulates the expression of a gene selected from pyk, nlpD, Cpn0585, or a gene belonging to the same regulatory or biosynthetic pathway as pyk, nlpD or Cpn0585, or a variant of said gene, or the that modulates the level and/or functional activity of an expression product of said gene. The method comprises contacting a preparation comprising said expression product (e.g., polypeptide or transcript), or a biologically active fragment thereof, or variant or derivative of these, or a genetic sequence that modulates the expression of said gene (e.g., the natural promoter relating to said gene), with a test agent, and detecting a change in the level and/or functional activity of said polypeptide or biologically active fragment thereof, or variant or derivative, or of a product expressed from said genetic sequence.

Modulators contemplated by the present invention includes agonists and antagonists of gene expression include antisense molecules, ribozymes and co-suppression molecules, as for example described in Section 2. Agonists include molecules which increase promoter activity or interfere with negative mechanisms. Agonists of a gene include molecules which overcome any negative regulatory mechanism. Antagonists of polypeptides encoded by a gene of interest include antibodies and inhibitor peptide fragments.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of a polypeptide according to the invention (especially Pyk, NlpD and CPn0585) are particularly preferred. In this regard, small molecules are particularly preferred because such molecules are more readily absorbed after oral administration, have fewer potential antigenic determinants, and/or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening for modulatory agents according to the invention can be achieved by any suitable method. For example, the method may include contacting a cell comprising a polynucleotide corresponding to a gene selected from pyk, nlpD, Cpn0585 or a gene belonging to the same regulatory or biosynthetic pathway as pyk, nlpD, Cpn0585, ompA, ompB, hsp60, or as a gene involved in the biosynthesis of LPS, or a variant of said gene, with an agent suspected of having said modulatory activity and screening for the modulation of the level and/or functional activity of a protein encoded by said polynucleotide, or the modulation of the level of a transcript encoded by the polynucleotide, or the modulation of the activity or expression of a downstream cellular target of said protein or said transcript. Detecting such modulation can be achieved utilising techniques including, but not restricted to, ELISA, cell-based ELISA, filter-binding ELISA, inhibition ELISA, Western blots, North Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR) and gel retardation assays.

It will be understood that a polynucleotide from which a target molecule of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g. a domain such as a protein binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilised, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than target molecule itself or the reporter polynucleotide operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression and/or activity of a target molecule according to the invention.

In a series of preferred embodiments, the present invention provides assays for identifying small molecules or other compounds (i.e., modulatory agents) which are capable of inducing or inhibiting the level and/or or functional activity of target molecules according to the invention. The assays may be performed in vitro using non-transformed cells, immortalised cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, β-galactosidase or luciferase) operatively linked to a target molecule-related gene regulatory region in a recombinant construct.

Thus, for example, one may culture cells which produce a particular target molecule and add to the culture medium one or more test compounds. After allowing a sufficient period of time (e.g., 6-72 hours) for the compound to induce or inhibit the level and/or functional activity of the target molecule, any change in said level from an established baseline may be detected using any of the techniques described above and well known in the art. In particularly preferred embodiments, the cells are epithelial cells. Using the nucleic acid probes and/or antigen-binding molecules disclosed herein, detection of changes in the level and or functional activity of a target molecule, and thus identification of the compound as agonist or antagonist of the target molecule, requires only routine experimentation.

In particularly preferred embodiments, a recombinant assay is employed in which a reporter polynucleotide encoding, for example, GFP, β-galactosidase or luciferase is operably linked to the 5′ regulatory regions of a target molecule related gene. Such regulatory regions may be easily isolated and cloned by one of ordinary skill in the art in light of the present disclosure of the coding regions of these genes. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modelling, and other routine procedures employed in rational drug design.

In another embodiment, a target molecule modulator can be identified by measuring the ability of a candidate agent to decrease the number of cells in an animal, which contain the persistent form of a species of a genus belonging to the family Chlamydiaceae. The animal is preferably a mammal such as a rabbit, gerbil, mouse, or rat. In this regard, reference may be made to Yang et al. (1993, Infection and Immunity 61: 2037-2040) and Fong et al. (1999, Infection and Immunity 67: 6048-6055), who describe a mouse model and a rabbit model, respectively for studying the pathogenesis of C. pneumoniae. In one embodiment of this method, a candidate agent is administered to the mammal, and the number of cells containing a said species in the persistent phase is determined using morphology based assays as, for example, described above. A compound tests positive if the number of cells containing persistent form(s) of said species in a sample taken from the animal to which the agent had been administered is less than that present in an equivalent sample from an untreated animal.

In yet another embodiment, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesised by any suitable technique. Such molecules may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, a target polypeptide according to the invention may be synthesised using solution synthesis or solid phase synthesis as described, for example, in Chapter 9 of Atherton and Shephard (supra) and in Roberge et al (1995, Science 269: 202).

To identify and isolate the peptide/solid phase support that interacts and forms a complex with a target molecule, preferably a target polypeptide, it is necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labelling with enzymes, fluorescent dyes or coloured or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-target polypeptide complex changes colour, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labelled epitope specific antigen-binding molecule. Once isolated, the identity of the peptide attached to the solid phase support may be determined by peptide sequencing.

4. Variant Polypeptides

The invention also contemplates the use and detection of variants of the polypeptide products of pyk, nlpD, Cpn0585, or of a gene belonging to the same regulatory or biosynthetic pathway as pyk, nlpD or Cpn0585, wherein the variants comprise an activity of a reference polypeptide of the invention. Variant or homologous polypeptides corresponding to other chlamydial isolates are known and it will be understood that such variant polypeptides are also encompassed by the present invention. Alternatively, variant polypeptides may be deduced from other species belonging to the family Chlamydiaceae by isolation of polynucleotide variants by standard protocols known in the art. In general, variants will be at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% homologous to a polypeptide as for example shown in any one of SEQ ID NO: 2, 4, 10, 16, 18, 22, 26, 32, 34 or 36, or in fragments thereof. Suitably, variants will have at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity to the sequence set forth in any one of SEQ ID NO: 2, 4, 10, 16, 18, 22, 26, 32, 34 or 36.

Variant peptides or polypeptides, resulting from rational or established methods of mutagenesis or from combinatorial chemistries, for example, may comprise conservative amino acid substitutions. Exemplary conservative substitutions in a polypeptide or polypeptide fragment according to the invention may be made according to TABLE B, supra.

5. Polypeptide Derivatives

With reference to suitable derivatives of the invention, such derivatives include amino acid deletions and/or additions to a polypeptide, fragment or variant of the invention, wherein said derivatives comprise an activity of a reference polypeptide of the invention (e.g., pyruvate kinase activity, inclusion membrane protein function). “Additions” of amino acids may include fusion of the polypeptides, fragments and polypeptide variants of the invention with other polypeptides or proteins. For example, it will be appreciated that said polypeptides, fragments or variants may be incorporated into larger polypeptides, and that such larger polypeptides may also be expected to have an activity of the parent polypeptide.

The polypeptides, fragments or variants of the invention may be fused to a further protein, for example, which is not derived from the original host. The further protein may assist in the purification of the fusion protein. For instance, a polyhistidine tag or a maltose binding protein may be used in this respect as described in more detail below. Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2, 4, 6-trinitrobenzene sulphonic acid (TNBS). The carboxyl group may be modified by carbodiimide activation via O-acylisourea formation followed by subsequent derivatisation, by way of example, to a corresponding amide. The guanidine group of arginine residues may be modified by formation of heterocyclic condensation products with reagents such as 2,3-butanedione, phenylglyoxal and glyoxal. Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH. Tryptophan residues may be modified, for example, by alkylation of the indole ring with 2-hydroxy-5-nitrobenzyl bromide or sulphonyl halides or by oxidation with N-bromosuccinimide. Tyrosine residues may be modified by nitration with tetranitromethane to form a 3-nitrotyrosine derivative. The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

6. Methods of Preparing a Polypeptide of the Invention

A polypeptide of the invention, or fragment thereof, or variant or derivative of these, may be prepared by any suitable procedure known to those of skill in the art. For example, a polypeptide may be prepared by a procedure including the steps of (a) preparing a recombinant polynucleotide comprising a nucleotide sequence encoding a polypeptide comprising the sequence set forth in any one of SEQ ID NO: 2, 4, 10, 16, 18, 22, 26, 32, 34 or 36, or a biologically active fragment thereof, or variant or derivative of these, which nucleotide sequence is operably linked to regulatory elements; (b) introducing the recombinant polynucleotide into a suitable host cell; (c) culturing the host cell to express recombinant polypeptide from said recombinant polynucleotide; and (d) isolating the recombinant polypeptide. Preferred nucleotide sequences include, but are not limited to the sequences set forth in SEQ ID NO: 1, 3, 9, 15, 17, 21, 25, 31, 33 or 35.

The recombinant polynucleotide is preferably in the form of an expression vector that may be a self-replicating extra-chromosomal vector such as a plasmid, or a vector that integrates into a host genome. The regulatory elements will generally be appropriate for the host cell used for expression. Numerous types of appropriate expression vectors and suitable regulatory sequences are known in the art for a variety of host cells. Typically, the regulatory elements include, but are not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter. Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags. In an especially preferred embodiment, the vector is pPROEx (Life Technologies).

The step of introducing into the host cell the recombinant polynucleotide may be effected by any suitable method including transfection, and transformation, the choice of which will be dependent on the host cell employed. Such methods are well known to those of skill in the art.

Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., 1989, in particular Sections 16 and 17; Ausubel et al., (1994-1998), in particular Chapters 10 and 16; and Coligan et al., (1995-1997), in particular Chapters 1, 5 and 6. Alternatively, the polypeptide, fragment, variant or derivative may be synthesised using solution synthesis or solid phase synthesis as described, for example, in Chapter 9 of Atherton and Shephard (supra) and in Roberge et al (1995).

7. Polynucleotides Variants

In general, polynucleotide variants according to the invention comprise regions that show at least 50%, preferably at least 55%, more preferably at least 60%, even more preferably at least 65%, even more preferably at least 70%, even more preferably at least 75%, even more preferably at least 80%, even more preferably at least 85%, even more preferably at least 90% and still even more preferably at least 95% sequence identity over a reference polynucleotide sequence of identical size (“comparison window”) or when compared to an aligned sequence in which the alignment is performed by a computer homology program known in the art. In accordance with the present invention, the reference polynucleotide sequence corresponds to a gene selected from pyk, nlpD, Cpn0585, or a gene belonging to the same regulatory or biosynthetic pathway as pyk, nlpD or Cpn0585.

What constitutes suitable variants may be determined by conventional techniques. For example, a polynucleotide according to any one of SEQ ID NO: 1, 3, 9, 15, 17, 21, 25, 31, 33 or 35 can be mutated using random mutagenesis (e.g., transposon mutagenesis), oligonucleotide-mediated (or site-directed) mutagenesis, PCR mutagenesis and cassette mutagenesis as is known in the art.

Alternatively, suitable polynucleotide sequence variants of the invention may be prepared according to the following procedure: creating primers which are optionally degenerate wherein each comprises a portion of a reference polynucleotide encoding a reference polypeptide or fragment of the invention, preferably encoding the sequence set forth in SEQ ID NO: 2, 4, 10, 16, 18, 22, 26, 32, 34 or 36; obtaining a nucleic acid extract from an organism, which is preferably an animal, and more preferably a mammal; and using said primers to amplify, via nucleic acid amplification techniques, at least one amplification product from said nucleic acid extract, wherein said amplification product corresponds to a polynucleotide variant.

Suitable nucleic acid amplification techniques are well known to the skilled artisan, and include polymerase chain reaction (PCR) as for example described in Ausubel et al. (supra); strand displacement amplification (SDA) as for example described in U.S. Pat. No. 5,422,252; rolling circle replication (RCR) as for example described in Liu et al., (1996) and International application WO 92/01813) and Lizardi et al., (International Application WO 97/19193); nucleic acid sequence-based amplification (NASBA) as for example described by Sooknanan et al., (1994); and Q-β replicase amplification as for example described by Tyagi et al., (1996).

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al. (1989) Chapters 8-12.

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled artisan will recognise that a number of factors influence hybridisation. The specific activity of radioactively labelled polynucleotide sequence should typically be greater than or equal to about 10⁸ dpm/mg to provide a detectable signal. A radiolabelled nucleotide sequence of specific activity 10⁸ to 10⁹ dpm/mg can detect approximately 0.5 pg of DNA. It is well known in the art that sufficient DNA must be immobilised on the membrane to permit detection. It is desirable to have excess immobilised DNA, usually 10 μg. Adding an inert polymer such as 10% (w/v) dextran sulphate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilised polynucleotide following washing. Washing ensures that the labelled polynucleotide is hybridised only to the immobilised polynucleotide with a desired degree of complementarity to the labelled polynucleotide.

It will be understood that polynucleotide variants according to the invention will hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridise to a reference polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘfor formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:

Tₘ=81.5+16.6 (log₁₀ M)+0.41 (%G+C)−0.63 (% formamide)−(600/length)

wherein: M is the concentration of Na⁺, preferably in the range of 0.01 molar to 0.4 molar; %G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5×SSC, 5× Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1%SDS solution for 12 min at 65-68° C.

Methods for detecting a labelled polynucleotide hybridised to an immobilised polynucleotide are well known to practitioners in the art. Such methods include autoradiography, phosphorimaging, and chemiluminescent, fluorescent and colorimetric detection.

8. Detection of the Persistent Phase of the Chlamydial Developmental Cycle and Diagnosis of Chronic Chlamydial Infections

The invention also features a method for detecting a species of a genus belonging to the family Chlamydiaceae in the persistent phase of its developmental cycle. The method comprises detecting, relative to the lytic phase of said developmental cycle, a change in the level and/or functional activity of an expression product of a gene selected from pyk, nlpD, Cpn0585, or a gene belonging to the same regulatory or biosynthetic pathway as pyk, nlpD or Cpn0585, or a variant of said gene.

The invention also encompasses a method for diagnosis of a persistent or chronic chlamydial infection in a patient by detecting in a biological sample obtained from said patient a change in the level and/or functional activity of a gene or expression product as described above. Conditions in which it would be particularly important to be able to diagnose persistent chlamydial infection include, but are not restricted to, cardiovascular diseases such as coronary artery disease, carotid artery disease, stroke, aneurisms; chronic respiratory diseases such as chronic obstructive pulmonary disease; chronic infertility problems in females such as tubal blockage); chronic eye infections (such as trachoma). Being able to diagnose the chronic state of chlamydial disease might enable alternate therapy directed at eliminating the persistent state of the chlamydial infection as, for example, described herein.

8.1 Nucleic Acid-Based Detection

One embodiment of the instant invention comprises a method for detecting the persistent phase or for diagnosis of a chronic chlamydial infection comprises qualitatively or quantitatively determining the level of transcript expressed by a gene selected from pyk, nlpD, Cpn0585, or a gene belonging to the same regulatory or biosynthetic pathway as pyk, nlpD or Cpn0585, or a variant of said gene. In a preferred embodiment, the level of said transcript is compared to a reference or baseline level of said transcript corresponding to the lytic phase of a chlamydial species. In these embodiments, nucleic acid can be isolated from cells contained in the biological sample, according to standard methodologies (Sambrook, et al., “Molecular Cloning. A Laboratory Manual”, Cold Spring Harbor Press, 1989; Ausubel et al., “Current Protocols in Molecular Biology”, John Wiley & Sons Inc, 1994-1998). The nucleic acid may be genomic DNA or fractionated or whole cell RNA. Where RNA is used, it may be desired to convert the RNA to a complementary DNA. The cell is preferably an epithelial cell including, but not limited to, an epithelial cell of the genital tract, respiratory tract, cardiovascular system, reproductive system (e.g., fallopian tubes) or conjunctiva or from arthritic joints. In one embodiment, the RNA is whole cell RNA; in another, it is poly-A RNA. In one embodiment, the nucleic acid is amplified by a nucleic acid amplification technique. Suitable nucleic acid amplification techniques are well known to the skilled person, and include the polymerase chain reaction (PCR) as for example described in Ausubel et al. (supra); strand displacement amplification (SDA) as for example described in U.S. Pat. No. 5,422,252; rolling circle replication (RCR) as for example described in Liu et al, (1996) and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93: 5395-5400).

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994, J Macromol. Sci. Pure, Appl. Chem., A31(1): 1355-1376).

Following detection, one may compare the results seen in a test sample with a control reaction corresponding to the lytic phase of the developmental cycle of a chlamydial species.

8.2 Protein-Based Detection

8.2.1 Antigen-Binding Molecules

Antigen-binding molecules that are immuno-interactive with a target molecule of the present invention can be used in measuring an increase or decrease in the expression of a gene selected from pyk, nlpD, Cpn0585, or a gene belonging to the same regulatory or biosynthetic pathway as pyk, nlpD or Cpn0585, or a variant of said gene. Thus, the present invention also contemplates antigen-binding molecules that bind specifically to a polypeptide encoded by those genes or to proteins that regulate or otherwise influence the level and/or functional activity of one or more said polypeptides. For example, the antigen-binding molecules may comprise whole polyclonal antibodies. Such antibodies may be prepared, for example, by injecting a target molecule (e.g., a persistent phase-associated polypeptide or portion thereof, or a lytic phase-associated polypeptide or portion thereof) of the invention into a production species, which may include mice or rabbits, to obtain polyclonal antisera. Methods of producing polyclonal antibodies are well known to those skilled in the art. Exemplary protocols which may be used are described for example in Coligan et al., “Current Protocols In Immunology”, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Kohler and Milstein (1975, Nature 256, 495-497), or by more recent modifications thereof as described, for example, in Coligan et al., (1991, supra) by immortalising spleen or other antibody-producing cells derived from a production species which has been inoculated with target molecule of the invention.

The invention also contemplates as antigen-binding molecules Fv, Fab, Fab′ and F(ab′)₂ immunoglobulin fragments. Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (Kreber et al. 1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plünckthun et al (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond therebetween. Suitable methods of producing dsFv are described for example in (Glockscuther et al. Biochem. 29: 1363-1367; Reiter et al. 1994, J. Biol. Chem. 269: 18327-18331; Reiter et al. 1994, Biochem. 33: 5451-5459; Reiter et al. 1994. Cancer Res. 54: 2714-2718; Webber et al. 1995, Mol. Immunol. 32: 249-258).

Also contemplated as antigen-binding molecules are single variable region domains (termed dAbs) as for example disclosed in (Ward et al. 1989, Nature 341: 544-546; Hamers-Casterman et al. 1993, Nature. 363: 446-448; Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (ie. having more than one antigen-binding site). Such multivalent molecules may be specific for one or more antigens. Multivalent molecules of this type may be prepared by dimerisation of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerisation may be facilitated by fusion of the antibody fragments to amphiphilic helices that naturally dimerise (Pack P. Plünckthun, 1992, Biochem. 31: 1579-1584), or by use of domains (such as the leucine zippers jun and fos) that preferentially heterodimerise (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

Also contemplated as antigen binding molecules are humanised antibodies. Humanised antibodies are produced by transferring complementary determining regions from heavy and light variable chains of a non human (e.g., rodent, preferably mouse) immunoglobulin into a human variable domain. Typical residues of human antibodies are then substituted in the framework regions of the non human counterparts. The use of antibody components derived from humanised antibodies obviates potential problems associated with the immunogenicity of non human constant regions. General techniques for cloning non human, particular murine, immunoglobulin variable domains are described, for example, by Orlandi et al. (1989, Proc. Natl. Acad. Sci. USA 86: 3833). Techniques for producing humanised monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies”, in Protein Engineering: Principles and Practice Cleland et al. (eds.), pages 399-434 (John Wiley & Sons, Inc. 1996), and by Queen et al., U.S. Pat. No. 5,693,762 (1997).

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra). The antigen-binding molecules can also be used to screen expression libraries for variant polypeptides of the invention as described herein. They can also be used to detect polypeptides, polypeptide fragments, variants and derivatives of the invention.

8.2.2 Immunodiagnostic Assays

The above antigen-binding molecules have utility in measuring directly or indirectly modulation of expression of a gene selected from pyk, nlpD, Cpn0585, or of a gene belonging to the same regulatory or biosynthetic pathway as pyk, nlpD or Cpn0585, or of a variant of said gene, through techniques such as ELISAs and Western blotting. Illustrative assay strategies which can be used to detect a target polypeptide of the invention include, but are not limited to, immunoassays involving the binding of an antigen-binding molecule to the target polypeptide (e.g., NlpD or Pyk) in the sample, and the detection of a complex comprising the antigen-binding molecule and the target polypeptide. Preferred immunoassays are those that can measure the level and/or functional activity of a target molecule of the invention. Typically, an antigen-binding molecule that is immuno-interactive with a target polypeptide of the invention is contacted with a biological sample suspected of containing said target polypeptide. The biological sample is suitably a specimen, which is suspected of containing a chlamydial organism in its persistent phase. For example, the biological sample may comprise sputums from chronic obstructive pulmonary diseases (COPD) patients, plaque from cardiovascular disease patients or fallopian tube washings from infertile women. The concentration of a complex comprising the antigen-binding molecule and the target polypeptide is measured and the measured complex concentration is then related to the concentration of target polypeptide in the sample. Consistent with the present invention, the concentration of said polypeptide is compared to a reference or baseline level of said polypeptide corresponding to the lytic phase of the developmental cycle of a chlamydial species under test. The presence of the persistent phase is detected or a chronic chlamydial infection is diagnosed if the concentration of the polypeptide corresponds to a non-reference level concentration.

Any suitable technique for determining formation of an antigen-binding molecule-target antigen complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to Coligan et al. (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4, 424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilised on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample is one that might contain an antigen including a tissue or fluid as described above.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs or nicroplates, or any other surface suitable for conducting an immunoassay. The binding processes are well known in the art and generally consist of cross-linking, covalently binding or physically adsorbing, the polymer-antibody complex to the solid support, which is then washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilised antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. Nos. 4,366,241, U.S. 4,843,000, and U.S. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al), 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodates. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemiluminescent or bioluminescent molecules may also be employed.

It will be well understood that other means of testing target polypeptide (e.g., Pyk, NlpD, CPn0585) levels are available, including, for instance, those involving testing for an altered level of the target polypeptide binding activity to the target polypeptide binding partner, or Western blot analysis of target protein levels in tissues, cells or fluids using anti-target protein antigen-binding molecules, or assaying the amount of antigen-binding molecule or other target polypeptide binding partner which is not bound to a sample, and subtracting from the total amount of antigen-binding molecule or binding partner added.

Alternatively, the presence of a chlamydial infection may be detected by assaying a patient's immune response to chlamydial antigens, particularly those antigens that are expressed at higher levels in, or whose presence is associated with, the persistent phase of the chlamydial developmental cycle. Components of the patient's immune system whose activity may be assayed include, but are not limited to, antibodies, B cells, T cells, dendritic cells and macrophages. For example, an immune response can be measured by standard tests including: direct measurement of peripheral blood lymphocytes by means known to the art; natural killer cell cytotoxicity assays (see, e.g., Provinciali M. et al (1992, J. Immunol. Meth. 155: 19-24), cell proliferation assays (see, e.g., Vollenweider, I. And Groseurth, P. J. (1992, J. Immunol. Meth. 149: 133-135), immunoassays of immune cells and subsets (see, e.g., Loeffler, D. A., et al. (1992, Cytom. 13: 169-174); Rivoltini, L., et al. (1992, Can. Immunol. Immunother. 34: 241-251); or skin tests for cell-mediated immunity (see, e.g., Chang, A. E. et al (1993, Cancer Res. 53: 1043-1050). CTL lysis assays may also be employed using stimulated splenocytes or peripheral blood mononuclear cells (PBMC) on peptide coated or recombinant virus infected cells using ⁵¹Cr or Alamar Blue™ labeled target cells. Such assays can be performed using for example primate, mouse or human cells (Allen et al., 2000, J. Immunol. 164(9): 4968-4978 also Woodberry et al., infra). In a preferred embodiment, the presence of a persistent chlamydial organism is detected by detecting antibodies to persistent phase antigens (i.e., whose presence or overexpression is associated with the persistent phase of the chlamydial developmental cycle). Suitably, such detection is facilitated by screening sera of a patient with a recombinant persistent phase antigen or portion thereof (e.g., by ELISA assay or by Western blot) for the presence of specific antibodies (IgG, IgM, IgA or IgE) that are immuno-interactive with that antigen or portion.

9. Therapeutic and Prophylactic Uses

The modulating agents of the invention prepared, for example, according to methods described in Section 3 supra have utility in compositions for treating and or preventing chlamydial infections, particularly chronic or persistent chlamydial infections. Accordingly, the present invention encompasses a method for treatment and/or prophylaxis of a chronic chlamydial infection by administering to a patient in need thereof an effective amount of agent, which specifically targets the persistent phase of the chlamydial developmental cycle, for a time and under conditions sufficient to treat and/or prevent the infection. In accordance with the present invention, the agent will modulate the expression of a gene or the level and/or functional activity of an expression product of said gene, wherein the gene is selected from pyk, nlpD, Cpn0585, or a gene belonging to the same regulatory or biosynthetic pathway as pyk, nlpD or Cpn0585, or a variant of said gene. In one embodiment, the agent is effective in killing or otherwise impairing or attenuating a chlamydial organism in the persistent phase of its developmental cycle.

In another embodiment, the agent is effective in causing said organism to revert or otherwise enter the lytic phase of its developmental cycle. In this embodiment, the invention contemplates the use of a second agent which modulates the expression of a gene associated with the lytic phase of said developmental cycle or the level and/or functional activity of an expression product of that gene. Indeed, a combination treatment which targets both the persistent state and also the lytic state is likely to be the most effective in eliminating chlamydial infection (particularly the chronic/persistent state) and hence substantially preventing chlamydial disease outcomes. Accordingly, the invention is also directed to a method for treating and/or preventing a chronic or lytic infection caused by chlamydial organism, comprising sequentially or simultaneously administering to a patient of a first agent and a second agent, wherein the first agent, which modulates the expression of a first gene expressed in the persistent phase of the developmental cycle of organism, or the level and/or functional activity of an expression product of said first gene, is administered to the patient for a time and under conditions sufficient to cause said organism to enter the lytic phase of said developmental cycle and wherein the second agent, which modulates the expression of a second gene associated with the lytic phase of said developmental cycle or the level and/or functional activity of an expression product of said second gene, is also administered to the patient for a time and under conditions sufficient to kill or otherwise inactivate or attenuate said organism.

The second agent is preferably an antibiotic that acts on actively replicating chlamydial organisms and that is, therefore, effective in killing or otherwise impairing or attenuating said chlamydial organism in the lytic phase of its developmental cycle. Any suitable antibiotics are contemplated by the present invention and include, but are not limited to, tetracycline, erythromycin, azithromycin, ofloxacin, ciprofloxin or prodrugs or analogues thereof.

The invention also envisions a composition for treatment and/or prophylaxis of chronic chlamydial infection, comprising a modulatory agent as broadly described above, together with a pharmaceutically acceptable carrier.

The modulatory agent(s) can be administered to a patient either by themselves, or in pharmaceutical compositions where they are mixed with suitable pharmaceutically acceptable carrier. Depending on the specific conditions being treated, modulatory agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines. Preferably, but not essentially, the composition is administered intranasally, orally and/or intragastrically and preferably in association with a mucosal adjuvant as for example described herein.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction or attenuation of a chlamydial infection. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the chronic chlamydial infection, the physician may evaluate fluid or tissue levels of a target molecule of the invention, and progression of the disorder. In any event, those of skill in the art may readily determine suitable dosages of the agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical preparations that can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the modulatory agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Modulating agents of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition of the activity or level of a target molecule of the invention). Such information can be used to more accurately determine useful doses in humans.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain target molecule-inhibitory effects or target molecule activating or stabilising effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m2/day, typically from 5-100 mg/m²/day.

Alternately, one may administer the compound in a local rather than systemic manner, for example, via injection of the compound directly into a tissue, often in a depot or sustained release formulation.

Furthermore, one may administer the drug in a targeted drug delivery system, for example, in a liposome coated with an epithelium-specific antibody. The liposomes will be targeted to and taken up selectively by a particular epithelium such as mucosal epithelium.

In cases of local administration or selective uptake, the effective local concentration of the agent may not be related to plasma concentration.

In an alternate embodiment, a polynucleotide encoding a modulatory agent of the invention may be used as a therapeutic or prophylactic composition in the form of a “naked DNA” composition as is known in the art. For example, an expression vector comprising said polynucleotide operably linked to a regulatory polynucleotide (e.g. a promoter, transcriptional terminator, enhancer etc) may be introduced into an animal where it causes production of a modulatory agent in vivo, particular in epithelial tissue. The modulatory agent in this instance may be an antisense molecule or ribozyme.

The step of introducing the expression vector into a target cell will differ depending on the intended use and species, and can involve one or more of non-viral and viral vectors, cationic liposomes, retroviruses, and adenoviruses such as, for example, described in Mulligan, R. C., (1993 Science 260: 926-932. Such methods can include, for example:

Local application of the expression vector by injection (Wolff et al., 1990, Science 247: 1465-1468), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of said protein.

General systemic delivery by injection of DNA, (Calabretta et al., 1993, Cancer Treat. Rev. 19: 169-179), or RNA, alone or in combination with liposomes (Zhu et al., 1993, Science 261: 209-212), viral capsids or nanoparticles (Bertling et al., 1991, Biotech. Appl. Biochem. 13: 390-405) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by said expression vector, or of cells responsive to said protein.

Injection or implantation or delivery by any means, of cells that have been modified ex vivo by transfection (for example, in the presence of calcium phosphate: Chen et al., 1987, Mole. Cell Biochem. 7: 2745-2752, or of cationic lipids and polyamines: Rose et al., 1991, BioTech. 10: 520-525), infection, injection, electroporation (Shigekawa et al., 1988, BioTech. 6: 742-751) or any other way so as to increase the expression of said polynucleotide in those cells. The modification can be mediated by plasmid, bacteriophage, cosmid, viral (such as adenoviral or retroviral; Mulligan, 1993, Science 260: 926-932; Miller, 1992, Nature 357: 455-460; Salmons et al., 1993, Hum. Gen. Ther. 4: 129-141) or other vectors, or other agents of modification such as liposomes (Zhu et al., 1993, Science 261: 209-212), viral capsids or nanoparticles (Bertling et al., 1991, Biotech. Appl. Biochem. 13 390-405), or any other mediator of modification. The use of cells as a delivery vehicle for genes or gene products has been described by Barr et al., 1991, Science 254: 1507-1512 and by Dhawan et al., 1991, Science 254 1509-1512. Treated cells can be delivered in combination with any nutrient, growth factor, matrix or other agent that will promote their survival in the treated subject.

The present invention also encompasses a method for treatment and/or prophylaxis of a chronic infection caused by an organism of the Chlamydiaceae family in a patient by administering to said patient an immunopotentiating agent selected from a proteinaceous molecule comprising at least a portion of a polypeptide, or variant or derivative thereof, associated with the persistent phase of the developmental cycle of said organism, or a polynucleotide from which said proteinaceous molecule is expressed. Examples of such persistent phase-associated antigens include, but are not restricted to, a polypeptide encoded by pyk, nlpD, Cpn0585, or a gene that is upregulated and belonging to the same regulatory or biosynthetic pathway as pyk, nlpD or Cpn0585, or a variant of said gene, or a biologically portion of said polypeptide, or an expression vector comprising a polynucleotide encoding a said polypeptide or fragment, operably linked to a transcriptional regulatory element.

The invention further contemplates a method for treatment and/or prophylaxis of a lytic or chronic infection caused by an organism of the Chlamydiaceae family in a patient by sequentially or simultaneously administering to said patient effective amounts of a first immunopotentiating agent and a second immunopotentiating agent. The first immunopotentiating agent is suitably selected from a first proteinaceous molecule comprising at least a portion of a polypeptide, or variant or derivative thereof, associated with the persistent phase of the developmental cycle of said organism, or a polynucleotide from which said first proteinaceous molecule is expressed. The second immunopotentiating agent is suitably selected from a second proteinaceous molecule comprising at least a portion of a polypeptide, or a variant or derivative thereof, associated with the lytic phase of said developmental cycle, or a polynucleotide from which said second proteinaceous molecule is expressed. Any suitable lytic phase antigens may be used in this regard. In a preferred embodiment, the lytic phase antigen is MOMP or a biologically active fragment thereof, or an expression vector comprising a polynucleotide encoding said MOMP or said fragment, operably linked to a transcriptional regulatory element.

Thus, the combination of a persistent phase antigen and a lytic phase antigen may be used as actives in the preparation of immunopotentiating compositions or vaccines. Such preparation uses routine methods known to persons skilled in the art. Exemplary procedures include, for example, those described in NEW GENERATION VACCINES (1997, Levine et al., Marcel Dekker, Inc. New York, Basel Hong Kong). Typically, immunopotentiating compositions are prepared as injectables, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient. Suitable excipients are, for example, water, saline, dextrose, glycerol, ethanol, or the like and combinations thereof. In addition, if desired, the immunopotentiating composition or vaccine may contain minor amounts of auxiliary substances such as wetting or emulsifying agents, pH buffering agents, and/or adjuvants that enhance the effectiveness of the composition.

A polypeptide, fragment, variant or derivative of the invention according to the invention can be mixed, conjugated or fused with other antigens, including B or T cell epitopes of other antigens. In addition, it can be conjugated to a carrier as described below.

When an haptenic peptide is used (i.e., a peptide which reacts with cognate antibodies, but cannot itself elicit an immune response), it can be conjugated with an immunogenic carrier. Useful carriers are well known in the art and include for example: thyroglobulin; albumins such as human serum albumin; toxins, toxoids or any mutant crossreactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a haptenic peptide can be coupled to a T cell epitope of a bacterial toxin, toxoid or CRM. In this regard, reference may be made to U.S. Pat. No. 5,785,973.

In addition, a polypeptide, fragment, variant or derivative of the invention may act as a carrier protein in vaccine compositions directed against an organism of the Chlamydiaceae family.

The immunopotentiating compositions of the invention may be administered as multivalent subunit compositions or vaccines in combination with other chlamydial immunogens such as MOMP. Alternatively, or additionally, they may be administered in concert with immunologically active antigens against other pathogenic species such as, for example, the pathogenic bacteria H. influenzae, M. catarrhalis, N. gonorrhoeae, E. coli, S. pneumoniae etc.

The immunopotentiating compositions may include an adjuvant as is well known in the art. Suitable adjuvants include, but are not limited to: surface active substances such as hexadecylamine, octadecylamine, octadecyl amino acid esters, lysolecithin, dimethyldioctadecylammonium bromide, N, N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives such as N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 1983A, referred to as MTP-PE); dimethylglycine, tuftsin; oil emulsions; and mineral gels such as aluminum phosphate, aluminum hydroxide or alum; Freunds incomplete adjuvant, Freunds complete adjuvant, tetanus toxoid, diphtheria toxoid, ISCOMS, QuilA, and RIBI, which contains three components extracted from bacteria, monophosphoryl lipid A, trehalose dimycolate and cell wall skeleton (MPL+TDM+CWS) in a 2% squalene/Tween 80 emulsion lymphokines, and QuilA. The effectiveness of an adjuvant may be determined for example by measuring the amount of antibodies resulting from the administration of the composition, wherein those antibodies are directed against one or more said chlamydial antigens or by measuring antigen specific T cell proliferation or cytolytic activity.

In a preferred embodiment, the immunopotentiating composition is administered via a mucosal route such as, but not limited to, orally, urogenitally or transdermally or combination of these. Accordingly, the adjuvant is preferably a mucosal adjuvant. Preferably, the mucosal adjuvant is cholera toxin or diphtheria toxin. Mucosal adjuvants other than cholera toxin or diphtheria toxin which may be used in accordance with the present invention include non-toxic derivatives of said toxins, such as the B sub-unit (CTB), chemically modified cholera or diphtheria toxin, or related proteins produced by modification of the cholera toxin or diphtheria toxin amino acid sequence. These may be added to, or conjugated with, the polypeptides, fragments, variants or derivatives of the invention. The same techniques can be applied to other molecules with mucosal adjuvant or delivery properties such as Escherichia coli heat labile toxin. Other compounds with mucosal adjuvant or delivery activity may be used such as bile; polycations such as DEAE-dextran and polyornithine; detergents such as sodium dodecyl benzene sulphate; lipid-conjugated materials; antibiotics such as streptomycin; vitamin A; and other compounds that alter the structural or functional integrity of mucosal surfaces. Other mucosally active compounds include derivatives of microbial structures such as MDP; acridine and cimetidine.

The immunogenic agents of the invention may be delivered in ISCOMS (immune stimulating complexes), ISCOMS containing CTB, liposomes or encapsulated in compounds such as acrylates or poly(DL-lactide-co-glycoside) to form microspheres of a size suited to adsorption by M cells. Alternatively, micro or nanoparticles may be covalently attached to molecules, which have specific epithelial receptors. The polypeptide, fragments, variant or derivative of the invention may also be incorporated into oily emulsions and delivered orally. An extensive though not exhaustive list of adjuvants can be found in Cox and Coulter (Cox and Coulter, 1992, Advances in adjuvant technology and application. In Animal Parasite Control Using Biotechnology. Edited by W. K. Yong. Published by CRC Press).

In another embodiment, the adjuvant is an antigen-presenting cell, preferably a dendritic cell, which presents a processed persistent phase or lytic phase antigen on its surface. Such adjuvants may be prepared by contacting an antigen-presenting cell with a persistent phase or lytic phase antigen for a time and under conditions sufficient to allow said antigen to be internalised and processed by said antigen-presenting cell for presentation to said B lymphocytes and said T lymphocytes. A variety of different strategies can be used for improving delivery of exogenous antigen to the endogenous processing pathway of antigen-presenting cells, particularly of dendritic cells. These methods include insertion of antigen in pH-sensitive liposomes (Zhou and Huang, 1994, Immunomethods, 4: 229-235), osmotic lysis of pinosomes after pinocytic uptake of soluble antigen (Moore et al., 1988, Cell, 54; 777-785), and coupling of antigens to potent adjuvants (Aichele et al., 1990, J. Exp. Med., 171: 1815-1820; Gao et al., 1991, J. Immunol., 147: 3268-3273; Schulz et al., 1991, Proc. Natl. Acad. Sci. USA, 88: 991-993; Kuzu et al., 1993, Euro. J. Immunol., 23: 1397-1400; and Jondal et al., 1996, Immunity 5: 295-302). Adjuvants (e.g., Freund's adjuvant) can also be used to assist in the internalisation and presentation of processed antigen onto the surface of the antigen-presenting cells.

The polypeptides, fragments, variants or derivatives of the invention may be expressed by attenuated viral hosts. A virus may be rendered substantially avirulent by any suitable physical (e.g., heat treatment) or chemical means (e.g., formaldehyde treatment). Ideally, the infectivity of the virus is destroyed without affecting the proteins that carry the immunogenicity of the virus. From the foregoing, it will be appreciated that attenuated viral hosts may comprise live viruses or inactivated viruses.

Attenuated viral or bacterial hosts which may be useful in a vaccine according to the invention may comprise viral vectors inclusive of adenovirus, cytomegalovirus and preferably pox viruses such as vaccinia (see for example Paoletti and Panicali, U.S. Pat. No. 4,603,112) and attenuated Salmonella strains (see for example Stocker, U.S. Pat. No. 4,550,081).

Live vaccines are particularly advantageous because they lead to a prolonged stimulus that can confer substantially long-lasting immunity. Thus, as an alternative to the delivery of immunogenic agents in the form of a therapeutic or prophylactic immunopotentiating composition, these agents may be delivered to the host using a live vaccine vector, in particular using live recombinant bacteria, viruses or other live agents, containing the genetic material necessary for the expression of the polypeptide, fragment, variant or derivative of the invention as a foreign antigen.

Multivalent immunopotentiating compositions or vaccines can be prepared from one or more organisms of the Chlamydiaceae family that express different persistent phase antigens or epitopes. In addition, epitopes of other pathogenic microorganisms can be incorporated into the compositions.

In a preferred embodiment, this will involve the construction of a recombinant vaccinia virus to express a nucleic acid sequence according to the invention. Upon introduction into a host, the recombinant vaccinia virus expresses the immunogenic agent, and thereby elicits a host CTL response. For example, reference may be made to U.S. Pat. No. 4,722,848, which describes vaccinia vectors and methods useful in immunisation protocols. A variety of other vectors useful for therapeutic administration or immunisation with the immunogenic agents of the invention will be apparent to those skilled in the art from the present disclosure.

In a further embodiment, a polynucleotide of the invention may be used as a vaccine in the form of a “naked DNA” vaccine as is known in the art. For example, an expression vector of the invention may be introduced into a mammal, where it causes production of a polypeptide in vivo, against which the host mounts an immune response as for example described in Barry, M. et al., (1995, Nature, 377:632-635). Thus, the invention also contemplates nucleic acid-based immunopotentiating compositions comprising an expression vector including a polynucleotide encoding an at least one antigen selected from persistent phase chlamydial antigens or lytic phase chlamydial antigens, wherein said polynucleotide is operably linked to a regulatory polynucleotide, together with a pharmaceutically acceptable carrier.

With regard to nucleic acid based compositions, all modes of delivery of such compositions are contemplated by the present invention. Delivery of these compositions to cells or tissues of an animal may be facilitated by microprojectile bombardment, liposome mediated transfection (e.g., lipofectin or lipofectamine), electroporation, calcium phosphate or DEAE-dextran-mediated transfection, for example. In an alternate embodiment, a synthetic construct may be used as a therapeutic or prophylactic composition in the form of a “naked DNA” composition as is known in the art. A discussion of suitable delivery methods may be found in Chapter 9 of CURRENT PROTOCOLS IN MOLECULAR BIOLOGY (Eds. Ausubel et al.; John Wiley & Sons Inc., 1997 Edition) or on the Internet site DNAvaccine.com. The compositions may be administered by intradermal (e.g., using panjet™ delivery) or intramuscular routes.

The immunopotentiating compositions will suitably elicit a B cell response and preferably a T cell response. Immunopotentiating compositions which produce a desired immune response can be evaluated using animal models of chlamydial infection (e.g., mouse for both urogenital and respiratory and cardiovascular infections; guinea pig for predominantly urogenital infections). The selected animal model is suitably be vaccinated (e.g., via several mucosal routes) using either full length recombinant proteins or portions thereof and boosted after 4-6 weeks. The immune response (preferably both antibody and cell mediated) is typically measured at weekly intervals. Generally, after periods of 8 weeks and 6 months, the vaccinated as well as unvaccinated control animals, are challenged with live Chlamydia. The immune responses (preferably both antibody and cell mediated) are continued to be measured at weekly intervals. Typically, several animals from each group are sacrificed and the status of disease evaluated, after 3 and 6 months and compared with unvaccinated controls.

In order that the invention may be readily understood and put into practical effect, particular preferred embodiments will now be described by way of the following non-limiting examples.

### EXAMPLES

### Example 1

Altered Morphological Forms Observed in IFN-δ-Treated C. pneumoniae Cultures

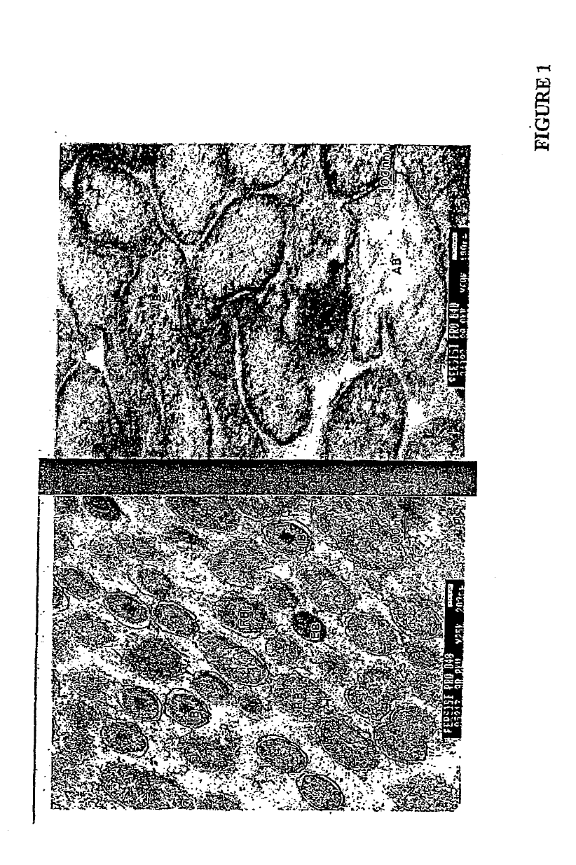

Normal (ie not treated with IFN-δ) cultures of C. pneumoniae IOL-207 contained characteristic membrane-bound inclusions approximately 5-8 μm in diameter, tightly packed with chlamydial particles. EBs were electron opaque, 200-400 nm, spherical-oval shaped particles with little periplasmic space and surrounded by an undulating cell membrane (FIG. 1a). RBs were round to oval in shape, 600-900 nm in diameter, with a typical electron translucent centre and condensed cytoplasm towards the periphery (FIG. 1a). By comparison, the IFN-δ-treated cultures, in addition to containing large numbers of morphologically normal inclusions with normal EBs and RBs, also contained 10-20% of forms exhibiting abnormal morphology (FIG. 1b). These abnormal inclusions were smaller (3.5 μm in diameter) than normal inclusions and contained considerably lower numbers of chlamydial particles. There were often pronounced extra-cellular spaces evident in these persistent inclusions. While the EBs in these inclusions appeared morphologically normal, the RBs were enlarged (400×900 nm) compared to those in normal inclusions (300×600 nm) and were pleomorphic, being either elongated with evidence of abnormal budding or branching occurring, or showing multi-layered membranes.

Methods

C. pneumoniae Cell Culture Conditions

HEp2 cells were grown in 75 cm² flasks at 37° C. in 5% CO₂ and maintained in complete DMEM consisting of Dulbecco's Minimum Essential Medium (Life Technologies) supplemented with 10% foetal bovine serum (CSL), 2 mM L-glutamine (Life Technologies), 100(g/mL streptomycin sulphate (Life Technologies) and 2 U/mL gentamycin (Life Technologies). C. pneumoniae IOL207 inoculum was generated by lysing 2×10⁷ infected cells (20-30% infected cell monolayer, 96 hours post-infection) in 20 ml SPG (0.22 M sucrose, 0.01 M phosphate, 0.0005 M L-glutamic acid) by the addition of 1 cm³ sterile glass beads followed by mechanical shaking plus bath sonication. The lysate was centrifuged at 1000 g for 5 minutes and the supernatant aliquoted and stored at −80° C.

C. pneumoniae infections for both RNA extraction and transmission electron microscopy (TEM) were established by replacing the growth medium of confluent Hep2 monolayers with 1 mL of chlamydial inoculum and 4 ml of complete DMEM followed by centrifugation at 1700 g for 30 minutes. The cells were subsequently incubated at 37° C. in 5% CO₂ for 6 hours, after which the inoculum was replaced with 10 ML complete DMEM containing 1 μg/mL cycloheximide in the presence (IFN-treated, I) or absence (untreated, N) of either 100 U/mL (for RNA extraction) or 10 U/mL (for TEM) of human interferon-gamma (Life Technologies). Cultures were grown for a further 18 hours (total of 24 hours post-infection) after which time half the samples were removed for analysis and half the samples had the media replaced (as above) and incubation continued until 48 hours post-infection. Samples for RNA extraction were washed twice with 5 ml Hanks buffered saline solution (Life Technologies) before the addition of 6 mL Tri-reagent (Sigma) and storage at −80° C. until RNA isolation. Samples for TEM were fixed in 3% glutaraldehyde in 0.1 M cacodylate buffer, osmotically adjusted to approximately 320 milliosmoles with sucrose and CaCl₂.

Transmission Electron Microscopy

Fixed samples for TEM were scraped from culture flasks and transferred to 1.5 ml micro-centrifuge tubes for further processing. After post-fixation in osmium tetroxide, followed by uranyl acetate, samples were dehydrated in increasing grades of ethanol (50, 70, 90%) and acetone (90, 100%) prior to infiltration and embedding in Spurr epoxy resin. Ultra-thin sections (approx. 90 nm) were collected onto 200 mesh copper grids and contrasted with 1% uranyl acetate and Reynold's lead citrate. Sections were examined and photographed using a JEOL 1200EX TEM operating at 80 kV.

### Example 2

Differential Gene Transcription in IFN-δ-Treated Versus Normal C. pneumoniae Cultures

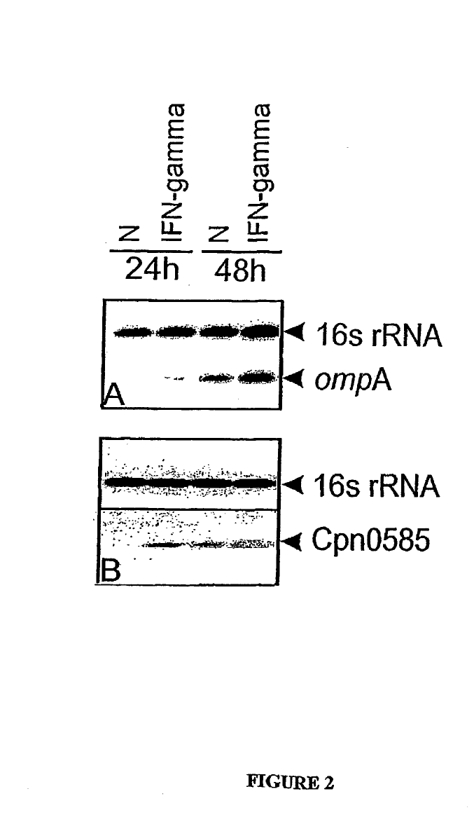

A total of 14 chlamydial genes (16SrRNA, ompA, ompB, omcB, 76 kDa, gseA, pmpl, gltX, hsp6O, yaeT, pyk, nlpD, Cpn0585, Cpn1046) were analysed by RT-PCR (and Southern blotting for the low transcript level genes) at 24 hour and 48 hours post-infection. Two genes (16SrRNA and gltX) were used as internal standards for relative comparison of gene expression between treated and non-treated cultures, at each time point. 16SrRNA was chosen for the highly transcribed genes and gltX for those genes with lower levels of transcription because 16SrRNA was thought to dominate the consumption of dNTPs from any low level transcribed gene in the same PCR reaction. In most cases, the levels of control transcript (either 16SrRNA or gltX) were equal (within 10%) between the same batches of normal and IFN-δ-treated cultures, enabling direct comparison of the test genes between normal and IFN-δ-treated cultures (FIG. 2). In the few instances where the levels of control transcript varied between normal and IFN-δ-treated cultures, the control levels were used to normalise the test gene results. The results for each gene were repeated in at least duplicate.

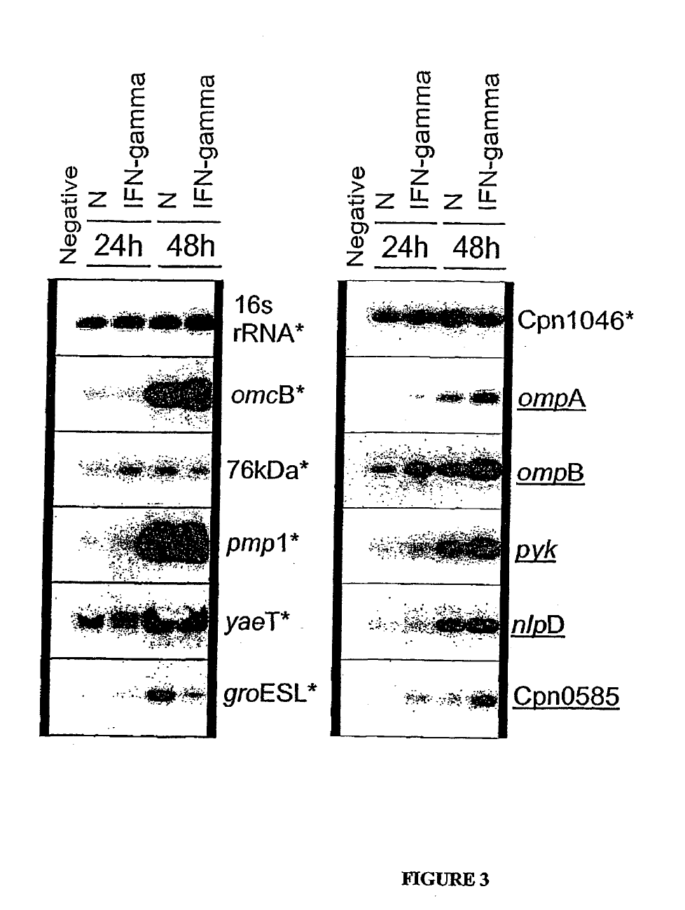

Nine genes (16SrRNA, omcB, 76 kDa, gseA, pmpl, gltX, hsp6O, yaeT, Cpn1046) showed approximately equal levels of transcription in normal and IFN-δ-treated cultures (FIG. 3; indicated with *). In comparison to these equally transcribed genes, five genes (ompA, ompB, pyk, nlpD, Cpn0585) clearly were transcribed at higher levels in the IFN-δ-treated cultures (FIG. 3). While pyk and nlpD genes showed repeatable upregulation, the differences between normal and IFN-δ-treated cultures was modest. In comparison, upregulation was more evident with ompA, ompB and particularly Cpn0585. At the 24 hour time point, there was no evidence of any Cpn0585 gene transcript in the normal culture whereas there was significant gene transcription evident in the IFN-δ-treated cultures. By 48 hours post-infection, some transcript was evident in the normal cultures, however the level in the IFN-δ-treated cultures was estimated to be at least 3-4 times greater.

While the intensity of the RT-PCR product may not directly reflect the actual level of gene expression, due to primer and PCR efficiency in addition to the relative starting copy number of the transcript being amplified, we were able to estimate the temporal expression of most genes, at least in relation to the 24 hour post-infection time point. Of the genes analysed, six (16SrRNA, 76 kDa, yaeT, ompB, gseA and Cpn1046) were strongly transcribed at the 24 hour time point, suggestive of early-transcribed genes, three (ompA, pyk and nlpD) were weakly transcribed at the 24 hour time point, and the remaining three (omcB, pmpl and Cpn0585) were primarily transcribed late in the development cycle (ie. mainly at the 48 hour time point).

Methods

Nucleic Acids

RNA was extracted from the samples stored in Tri-reagent (above) and contaminating DNA removed by resuspending the RNA in 130 μL 10 mM Tris-Cl (pH 7.5), 10 mM MgCl₂, 1 mM dithioerythritol (DTE), 40 U RNase inhibitor treatment with 20 U RNase-free DNase 1 (Roche) for 30 minutes at 37° C. The RNA was further purified by processing through RNeasy mini columns (Qiagen) where 25-75 μg RNA was eluted in 50 μL of ddH₂O. Genomic DNA was extracted from C. pneumoniae IOL207 infected HEp2 cells (10⁷) following the TRI-reagent procedure for DNA extraction (Life Technologies) yielding 100 μg total DNA. The sequences of the primers used for PCR and RT-PCR analysis were synthesised by either Life Technologies (16SrRNA, ompA, omcB, ompB, pmpl, gltX, pyk) or Pacific Oligos (76 kDa, yaeT, nlpD, groESL, Cpn0585, Cpn1046).

Analysis of Gene Expression

10 μg of total RNA was primed with 1.0 μg of random hexamers (Roche) to generate cDNA following the method previously described (Mathews et al., 1999). cDNA samples were stored at approximately 50 ng/μL in 10 μL aliquots at −20° C. to limit freeze/thawing. Aliquots in use were stored at 4° C. between PCR assays. The presence of contaminating genomic DNA was excluded by performing PCR on RNA samples using the 16S rRNA primers.

A duplex PCR consisting of the gene of interest (Table 1) with an internal reference gene (either 16SrRNA or gltX) was performed. 251L PCR reactions contained 1×PCR buffer containing 1.5 mM MgCl₂ (Roche), 1 μM of each primer, 2 mM of each dNTP (Roche), 2U Taq polymerase (Roche) and 1 μL template (either cDNA, genomic DNA or TE). PCR conditions were 94° C. for 3 minutes followed by either 35 cycles (16SrRNA reference gene) or 40 cycles (gltX reference gene) of 94° C. 30 seconds, 53° C. 30 seconds and 72° C. 45 seconds with a final extension at 72° C. for 7 minutes in a Peltier PTC-200 thermal cycler (MJ Research, Watertown, Mass., USA). PCR products were electrophoresed through a 2% TBE (45 mM Tris-borate, 1 mM EDTA) agarose gel containing 1 μg/mL ethidium bromide.

Southern Blot Analysis

Probes were generated in 50 μL reactions by incorporating 0.2 mM DIG-dNTPs (Roche) into PCR reactions (primers Table 1, conditions as above). DNA was transferred to positively-charged nylon membranes (Roche) in 0.4M NaOH for 2 hours by capillary action and the blots rinsed in 2×SSC (20×SSC is 3M NaCl, 0.3 M NaCitrate) and UV fixed for 2 minutes. Blots were pre-hybridised for 30 minutes at 42° C. with DIG-Easy Hyb solution (Roche) before the addition of 6-10 μL probe followed by hybridisation at 42° C. overnight. After hybridisation, the blots were washed and detected with CDP-Star (Roche) according to the manufacturer's instructions. The blots were exposed to Kodak X-ray film for 5 sec, 15 sec, 30 sec, 1 min, 5 min, 20 min and overnight exposures. A reference blot for quantification of band intensity was generated by Southern transfer and detection of 2-fold serial dilutions of omcB positive control PCR product as described above.

Discussion of Examples 1 and 2

A common feature of many chlamydial infections is that they are often asymptomatic and may persist for long periods of time if left untreated. It is likely that this inability of the host to clear the chlamydial infection enables the organism to establish a chronic state, which eventually leads to the resultant adverse immunopathology. What is unknown however, is whether these chronic/persistent chlamydial infections trigger the immune system in such a way as to induce adverse immunopathology. At appropriate concentrations, IFN-δ has been shown to inhibit the growth of C. trachomatis, C. psittaci and C. pneumoniae (see Beatty et al., 1994). The mechanism by which this occurs is thought to be via the induction of host cell indoleamine 2,3-dioxygenase, which results in the depletion of the host cell's tryptophan pool and a resultant nutrient deprivation for the chlamydiae. While the effects of various stress conditions (e.g., direct nutrient starvation, IFN-δ treatment, penicillin treatment) have been well studied in the C. trachomatis system, very little has been done with C. pneumoniae. The current disclosure is the first to use IFN-δ treatment of C. pneumoniae and to demonstrate morphologically abnormal, persistent forms in this species. Very recently, Wolf et al. (2000) also reported the induction of abnormal forms of C. pneumoniae using ampicillin treatment, with RB morphology similar to that observed in our study using IFN-δ.

It is possible that the persistent phase of the chlamydial developmental cycle might be induced by a range of triggers, each resulting in growth-restricted aberrant chlamydial development. As suggested by Wolf et al. (2000) such growth restrictions might be more common in vivo than previously thought, making this phase crucial in vivo. If such growth-restricted persistent phases do occur regularly, then it might be expected that the organism would have an altered gene expression profile. As a preliminary study, the present inventors selected 14 genes for analysis. The gene transcription analyses were normalised to facilitate an accurate comparison between treatment groups on a gene-by-gene basis. While no obviously down-regulated genes were found, five of the 14 genes were found to be significantly and reproducibly upregulated in IFN-δ-treated cultures.

Two of these genes, ompA and ompB, are structural proteins thought to be important in cell wall rigidity. Disregulated expression of such proteins might explain the aberrant RB morphology observed in persistent cultures, particularly the multi-membranous forms seen in 48-hour IFN-δ cultures. The enzyme pyruvate kinase (pyk) was chosen for analysis because it catalyses the final step in glycolysis, from phosphoenolpyruvate to pyruvate with the release of ATP. The fact that pyk was upregulated in IFN-δ-treated cultures might suggest that under stress conditions, C. pneumoniae requires the release of stored energy. Cpn0585 was the most upregulated gene identified and this gene has a homologue, incA, in both C. trachomatis and C. psittaci whose protein product has been localised to the chlamydial inclusion membrane (Bannantine et al., 1998). It is likely that its role is either; (1) to ensure that individual inclusions fuse during chlamydial growth (although C. pneumoniae inclusions apparently do not fuse) or (2) to act as a porin to obtain nutrients from the host cell but presumably also to export key chlamydial proteins into its host cell, thereby influencing the ongoing infection. The gene incA is one of three inc genes, A, B and C, in the chlamydial genome (Stephens et al., 1999) and has very recently been shown to be required for fusion of C. trachomatis inclusions (Suchland et al., 2000). Upregulation of IncA (Cpn0585) in persistent C. pneumoniae cultures is, therefore, of particular interest and suggests that this pathogen has mechanisms for modulating its survival when under stressful conditions (IFN-δ-induced persistence; macrophage infection).

The other interesting gene that was upregulated in the persistent phase was nlpD. The C. pneumoniae nlpD gene product has significant homology with a major extracellular protein family, p60, from organisms such as Listeria monocytogenes (Bubert et al., 1992), Enterococcus faecalis and Bacillus (Margot et al., 1998). In these microorganisms, the protein has two functional domains, an N-terminal domain that contains repeated motifs thought to be responsible for binding to peptidoglycan and a C-terminal domain that has different activities depending on the organism, but usually is associated with key catalytic activities (eg peptidase). In Listeria, the homologous gene, iap (invasion associated protein) has been shown to be required for adherence to and invasion of nonphagocytic cells (eg fibroblasts) by this pathogen (Bubert et al., 1992). The C. pneumoniae nlpD gene product also has similar features to its P60 homologues. It has a 114 amino acid region at its N-terminal end that displays approximately 40% identity to the peptidoglycan-binding motif seen in p60 family proteins. It also has a region at its C-terminal that shows most similarity to the Enterococcus amidase. As with the other p60 proteins, it also has a cleavable N-terminal signal sequence. The involvement of nlpD in chlamydial pathogenesis is uncertain, however its upregulation in IFN-δ-induced persistence is of particular interest.

Messenger RNA transcript levels were measured, rather than protein levels, making it difficult to directly compare the present results to earlier reports using immunostaining. Nevertheless, it is interesting to note that no significant upregulation of the hsp60 gene was observed, whereas others have reported increased staining with anti-Hsp60 antibodies in cells from IFN-δ-induced persistent C. trachomatis cultures (Beatty et al., 1993a). These same authors also reported a reduced staining of persistent C. trachomatis RBs with anti-MOMP antibodies, whereas the present inventors found a significant upregulation of ompA gene transcripts. As observed by Matsumoto & Manire (1970) with penicillin treatment of C. psittaci cultures, multi-membraned forms were often present in stressed chlamydial cultures. These multi-membranous structures, which were also observed in the present study, probably contain abnormally formed surface structural proteins. It is possible that while there is some upregulation of ompA at the mRNA level, the protein may not be properly folded and or presented at the RB surface. This might explain the abnormal RB morphology that is commonly observed as well as the ineffective anti-MOMP antibody staining reported by Beatty et al. (1993a).

It is clear from the present study that C. pneumoniae can be induced to produce a proportion of morphologically abnormal persistent forms in vitro. These persistent forms have a considerably altered gene transcription profile that might represent a generic stressed state.

### Example 3

Preparation of Antibodies Specific to CPn0585 or NlpD

Short stretches of amino acids forming suitable peptide immunogens can be selected from a target gene/protein using standard methods or computer algorithms known in the art. For example reference may be made to Pellequer, J.-L., Westhof, E. and van Regenmortel, M. H. V. (1994) Epitope predictions from the primary structure of proteins. In Peptide antigens: a practical approach, pp7-25, Ed. Wisdom, G. B. (Oxford). Several short peptides have been designed for NlpD and CPn0585 as follows:

Two peptides have been designed, which relate to protein AAD18724 corresponding to CPn0585 (SEQ ID NO: 2):

1. A peptide consisting of the N-terminal and C-terminal sequences with a Cys residue between them (the combined N+C peptide) and having the following sequence:

Met-Ala-Thr-Pro-Ala-Gln-Lys-Ser-Cys-Arg-Leu-Glu-Gln-Glu-Gln-Phe-Gln-Gly [SEQ ID NO: 37]

N-Terminus H-; C-Terminus -OH
Length 18; Mass 10 mg
Molecular Wt 2052.3; Hydrophobicity 0.09
Conjugation 5 mg; Carrier Diphtheria Toxoid
Linker Maleimidocaproyl-N-Hydroxysuccinimide (MCS)

10 2. A peptide which was positive in PREDITOP and is both hydrophilic, and has a natural Cys at the C-terminal end of the sequence, which can be used for conjugation purposes, and has the following sequence:

Thr-Val-Gln-Asp-Leu-Arg-Ser-Arg-Ile-Asp-Asp-Glu-Gln-Lys-Arg-Cys [SEQ ID NO: 38]

N-Terminus H-; C-Terminus -NH2
Length 16; Molecular Wt 1961.2
Hydrophobicity −0.14; Charge +1 (+5 −4)
Conjugation 5 mg; Carrier Diphtheria Toxoid
Linker Maleimidocaproyl-N-Hydroxysuccinimide (MCS)

Two peptides have been designed, which relate to protein AAD19040, corresponding to NlpD as follows:

1. A peptide consisting of the combined N-terminal and C-terminal sequences with a Cys residue between them (the combined N+C peptide)

Met-Asn-Arg-Arg-Asp-Met-Val-Cys-Pro-Gly-Asp-Gln-Leu-Arg-Ile-Arg [SEQ ID NO: 39]

N-Terminus H-; C-Terminus -OH
Length 16; Mass 10 mg
Molecular Wt 1960.4; Hydrophobicity 0.07
Conjugation 5 mg; Carrier Diphtheria Toxoid
Linker Maleimidocaproyl-N-Hydroxysuccinimide (MCS)

10 2. The peptide with the highest value from the PREDITOP predictive method, which lacks a natural Cys in the sequence, but to which is added a terminal Cys for conjugation purposes, and has the following sequence:

Val-Thr-Ser-Lys-Arg-Ile-Gly-Val-Lys-Asp-Tyr-Asp-Glu-Gly-Phe-Cys [SEQ ID NO: 40]

N-Terminus H-; C-Terminus -NH2
Length 16; Mass 10 mg
Molecular Wt 1816.1; Hydrophobicity 0.11
Conjugation 5 mg; Carrier Diphtheria Toxoid
Linker Maleimidocaproyl-N-Hydroxysuccinimide (MCS)

These peptides will be used to immunise rabbits according to standard methods and antisera or antibodies derived therefrom used to diagnose chronic disease and persistent infection.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

TABLE I

Primer pairs and PCR product size for the genes under investigation.

| | | |
|---|---|---|
| | | PCR |
| | | Product |
| Gene | Primers | (bp) |

| | | | | |
|---|---|---|---|---|
| 16S | C16S-F2 | 5′-GGA TTT ATT GGG CGT AAA GG | [SEQ ID NO: 41] | 290 |
| rRNA | Ct16S-R | 5′-TCC ACA TCA AGT ATG CAT CG | [SEQ ID NO: 42] | |
| OmpA | CpnompA-F | 5′-GCTGCAAACTATACTACTGC | [SEQ ID NO: 43] | 125 |
| | CpnompA-R | 5′-GAAAACATCAAAGCGATCCC | [SEQ ID NO: 44] | |
| OmpB | CpnompB-F | 5′-GTGATGGGAAATTAGTCTGG | [SEQ ID NO: 45] | 212 |
| | CpnompB-R | 5′-ATC CTG TGT TCA CTA CTT CG | [SEQ ID NO: 46] | |
| OmcB | CpnompB-F | 5′-AGCAGAAGTTTACTCTGTCG | [SEQ ID NO: 47] | 242 |
| | CpnomcB-R | 5′-CTACTGATGGAAACCTAAGC | [SEQ ID NO: 48] | |
| 76kDa | Cpn76kDa-F | 5′-AAGATATCAAGGCTACTGATGAGGAAACCG | [SEQ ID NO: 49 ] | 255 |
| | Cpn76kDa-R | 5′-TTGATATCTAGAACTTGCTGCAGCGGGA | [SEQ ID NO: 50] | |
| pmp 1 | Cpnpmp1-F | 5′-GACTACTGCTATAGGTAAGG | [SEQ ID NO: 51] | 165 |
| | Cpnpmp1-R | 5′-GAGATGCTAAGTTTCCTAGC | [SEQ ID NO: 52] | |
| GltX | CpngltX-F | 5′-TCTCTTTCGTCCATTGATCG | [SEQ ID NO: 53] | 125 |
| | CpngltX-R | 5′-CTCAGGATTGTTAGAGTACC | [SEQ ID NO: 54] | |
| GroELS | Cpnhsp60B-F | 5′-GTCCAGTGAAATCATGGCCG | [SEQ ID NO: 55] | 298 |
| | Cpnhsp60AI-R | 5′-CCCATGTTTTCATGTTTGTC | [SEQ ID NO: 56] | |
| YaeT | CpnyaeT-F | 5′-TCAGGAAATCAAGTCGTTCC | [SEQ ID NO: 57] | 253 |
| | CpnyaeT-R | 5′-AGATTCCTGAGAACGTAAGC | [SEQ ID NO: 58] | |
| Pyk | Cpnpyk-F | 5′-TGTTGTTGTCTCTTCAGAGG | [SEQ ID NO: 59] | 152 |
| | Cpnpyk-R | 5′-CTACCCCAAACTTAAGATCC | [SEQ ID NO: 60] | |
| NIpD | CpnnlpD-F | 5′-TCAATGATCTTACCACCACC | [SEQ ID NO: 61] | 164 |
| | CpnnlpD-R | 5′-GTTACGCAATGCTATFGTCC | [SEQ ID NO: 62] | |
| Cpn0585 | Cpn0585-F | 5′-TGCATCTTATCAAGAGCTCG | [SEQ ID NO: 63] | 267 |
| | Cpn0585-R | 5′-GAAGTTAGCGGATTT7AGAGG | [SEQ ID NO: 64] | |
| Cpn1046 | Cpn1046-F | 5′-GAGGAGAACTGATAAGAACG | [SEQ ID NO: 65] | 269 |
| | Cpn1046-R | 5′-CTTAACTCCTGATCTCATCC | [SEQ ID NO: 66] | |

### BIBLIOGRAPHY

Bannantine, J.P., W. E. Stamm, R. J. Suchland, and D. D. Rockey. 1998. Chlamydia trachomatis IncA is localised to the inclusion membrane and is recognised by antisera from infected humans and primates. Infection & Immunity 66:6017-6021.

Beatty, W. L., G. I. Byrne, and R. P. Morrison. 1993a. Morphological and antigenic characterisation of interferon mediated persistent Chlamydia trachomatis infection in vitro. Proc Nat Acad Sci USA 90:3998-4002.

Beatty, W. L., R. P. Morrison, and G. I Byrne. 1993b. Persistent chlamydiae: from cell culture to a paradigm for chlamydial pathogensis. Microbiological reviews 58:686-699.

Beatty, W. L., R. P. Morrison, and G. I Byrne. 1994. Persistent chlamydiae: from cell culture to a pradigm for chlamydial pathogenesis. Microbiol. Reviews 58:686-699.

Beatty, W. L., R. P. Morrison, and G. I Byrne. 1995. Reactivation of persistent Chlamydia trachomatis infection in cell culture. Infect. Immun. 63: 199-205.

Bubert, A., M. Kuhn, W. Goebel, and S. Kohler. 1992. Structural and functional properties of the p60 proteins from different Listeria species. J Bact 174:8166-8171.

Coles, A. M., D. J. Reynolds, A. Harper, A. Devitt, and J. H. Pearce. 1993. Low-nutrient induction of abnormal chlamydial development: a novel component of chlamydial pathogenesis? FEMS Microbiol. Lett. 106: 193-200.

Harper, A., C. Pogson, M. Jones, M., and J. Pearce. 2000. Chlamydial development is adversely affected by minor changes in amino acid supply, blood plasma amino acid levels and glucose deprivation. Infection & Immunity 68:1457-1464.

Kalman, S., W. Mitchell, R. Marethe, C. Lammel, J. Fan, R. W. Hayman, L. Olinger, J. Grimwood, R. W. Davis, and R. S Stephens. 1999. Comparative genomes of Chlamydia pneumoniae and C. trachomatis. Nature Genetics 21:385-389.

Kramer, M. J., and F. B. Gordon. 1971. Ultrastructural analysis of the effects of penicilin and chlortetracycline on the development of a genital tract Chlamydia. Infect. Immun. 3: 333-341.

Margot, P., M. Wahlen, A. Gholamhuseinian, P. Piggot, and D. Karamata. 1998. The lytE gene of Bacillus subtilis 168 encodes a cell wall hydrolase. J Bacteriology 180:749-752.

Mathews, S., K. Volp, and P. Timms. 1999. Development of a quantitative gene expression assay for Chlamydia trachomatis identified temporal expression of sigma factors. FEBS Letters 458:354-358.

Matsumoto, A., and G. P. Manire. 1970. Electron microscopic observations on the effects of penicillin on the morphology of C. psittaci. J. Bacteriol. 101: 278-285.

Morrison, R. P., K. Lyng, and H. D. Caldwell. 1989. Chlamydial disease pathogenesis. Ocular hypersensitivity elicited by a genus-specific 57-kD protein. J Exp Medicine 169:663-675.

Read, T. D., R. C. Brunham, C. Shen, S. R. Gill, J. F. Heidelberg, et al. 2000. Genome sequences of Chlamydia trachomatis MoPn and Chlamydia pneumoniae AR39. Nucleic Acids res. 28: 1397-1406.

Stephens, R. S., S. Kalman, C. Lammel, J. fan, R. Marathe, L. Aravind, W. Mitchell, L. Olinger, R. L. Tatusov, Q. Zhao, E. V. Koonin, and R. W. Davis. 1999. Genome sequence of an obligate intracellular pathogen of humans: Chlamydia trachomatis. Science 282:754-759.

Suchland, R. J., D. D. Rockey, J. P. Bannantine, and W. E. Stamm. 2000. Isolates of Chlamydia trachomatis that occupy nonfusogenic inclusions lack IncA, a protein localised to the inclusion membrane. Infection & Immunity 68:360-367.

Wuenscher, M. D., S. Kohler, A. Bubert, U. Gerike, and W. Goebel. 1993. The iap gene of Listeria monocytogenes is essential for cell viability and its gene product, p60, has bacteriolytic activity. J Bact 175:3491-3501.

Wolf, K., E. Fischer, and T. Hackstadt. 2000. Ultrastructural analysis of developmental events in Chlamydia pneumoniae-infected cells. Infect. Immun. 68: 2379-2385.

1. A method for detecting an organism of the Chlamydiaceae family in the persistent phase of its developmental cycle, said method comprising detecting, relative to the lytic phase of said developmental cycle, a change in the level or functional activity of an expression product of a gene selected from pyk, nlpD, Cpn0585, or a gene belonging to the same regulatory or biosynthetic pathway as pyk, nlpD or Cpn0585.

2. The method of claim 1, wherein said change is an at least 10% change in said level or functional activity.

3. The method of claim 2, wherein the gene belonging to the same regulatory or biosynthetic pathway as pyk is selected from mrsA, pfkA_{—}1, pfkA_{—}2, dhnA, gapA, pgk, eno, pgmA, pgm, pgi or tpiS.

4. The method of claim 2, wherein the gene belonging to the same regulatory or biosynthetic pathway as nlpD is selected from amiA, murE, pbp3, yabC, murA, dacF, pbpB, amiB, glmU, murF, mraY, murD, murG, murC, ddlA, glmS or murB.

5. The method of claim 2, wherein the gene belonging to the same regulatory or biosynthetic pathway as Cpn0585 is selected from incA, incB, incC or Cpn0186.

6. The method of claim 1, wherein said gene is selected from pyk, nlpD or Cpn0585.

7. The method of claim 6, wherein pyk comprises the sequence set forth in SEQ ID NO: 9, 17, 21 or 31.

8. The method of claim 6, wherein the expression product of pyk is a transcript encoded by the sequence set forth in SEQ ID NO: 9, 17, 21 or 31.

9. The method of claim 6, wherein the expression product of pyk is a polypeptide comprising the sequence set forth in SEQ ID NO: 10, 18, 22 or 32.

10. The method of claim 6, wherein nlpD comprises the sequence set forth in SEQ ID NO: 3, 15, 25 or 35.

11. The method of claim 10, wherein the expression product of nlpd is a transcript encoded by the sequence set forth in SEQ ID NO: 3, 15, 25 or 35.

12. The method of claim 10, wherein the expression product of nlpD is a polypeptide comprising the sequence set forth in SEQ ID NO: 4, 16, 26 or 36.

13. The method of claim 6, wherein Cpn0585 comprises the sequence set forth in SEQ ID NO: 1 or 33.

14. The method of claim 13, wherein the expression product of Cpn0585 is a transcript encoded by the sequence set forth in SEQ ID NO: 1 or 33.

15. The method of claim 13, wherein the expression product of Cpn0585 is a polypeptide comprising the sequence set forth in SEQ ID NO: 2 or 34.

16. A method for diagnosis of a persistent or chronic infection in a patient, wherein said infection is caused by an organism of the Chlamydiaceae family, said method comprising detecting in a biological sample obtained from said patient, relative to the lytic phase of the developmental cycle of said organism, a change in the level or functional activity of an expression product of a gene selected from pyk, nlpD, Cpn0585, or a gene belonging to the same regulatory or biosynthetic pathway as pyk, nlpD or Cpn0585.

17. The method of claim 16, wherein said change is an at least 10% change in said level or functional activity.

18. The method of claim 17, wherein the gene belonging to the same regulatory or biosynthetic pathway as pyk is selected from mrsA, pfkA_{—}1, pfkA_{—}2, dhnA, gapA, pgk, eno, pgmA, pgm, pgi or tpiS.

19. The method of claim 17, wherein the gene belonging to the same regulatory or biosynthetic pathway as nlpD is selected from amiA, murE, pbp3, yabC, murA, dacF, pbpB, amiB, g/mU, murF, mraY, murD, murG, murC, ddlA, glmS or murB.

20. The method of claim 17, wherein the gene belonging to the same regulatory or biosynthetic pathway as Cpn0585 is selected from incA, incB, incC or Cpn0186.

21. The method of claim 16, wherein said gene is selected from pyk, nlpD or Cpn0585.

22. The method of claim 21, wherein pyk comprises the sequence set forth in SEQ ID NO: 9, 17, 21 or 31.

23. The method of claim 22, wherein the expression product of pyk is a transcript encoded by the sequence set forth in SEQ ID NO: 9, 17, 21 or 31.

24. The method of claim 22, wherein the expression product of pyk is a polypeptide comprising the sequence set forth in SEQ ID NO: 10, 18, 22 or 32.

25. The method of claim 21, wherein nlpD comprises the sequence set forth in SEQ ID NO: 3, 15, 25 or 35.

26. The method of claim 25, wherein the expression product of nlpD is a transcript encoded by the sequence set forth in SEQ ID NO: 3, 15, 25 or 35.

27. The method of claim 25, wherein the expression product of nlpD is a polypeptide comprising the sequence set forth in SEQ ID NO: 4, 16, 26 or 36.

28. The method of claim 21, wherein Cpn0585 comprises the sequence set forth in SEQ ID NO: 1 or 33.

29. The method of claim 28, wherein the expression product of Cpn0585 is a transcript encoded by the sequence set forth in SEQ ID NO: 1 or 33.

30. The method of claim 28, wherein the expression product of Cpn0585 is a polypeptide comprising the sequence set forth in SEQ ID NO: 2 or 34.

31. The method of claim 16, further comprising:
contacting the biological sample with an antigen-binding molecule that is immuno-interactive with a polypeptide expressed from said gene;
measuring the concentration of a complex comprising said polypeptide and the antigen binding molecule in said contacted sample; and
relating said measured complex concentration to the concentration of said polypeptide in said sample.

32. The method of claim 31, wherein the concentration of said polypeptide in said biological sample is compared to a reference level of said polypeptide corresponding to said lytic phase.

33. The method of claim 16, further comprising:
measuring the level of a transcript expressed from said gene in said biological sample.

34. The method of claim 33, wherein the level of said transcript in said biological sample is compared to a reference level of said transcript corresponding to said lytic phase.

35. The method of claim 16, further comprising:
contacting the biological sample with an antigen corresponding to at least a portion of a polypeptide encoded by said gene;
measuring the concentration of a complex comprising said antigen and an antigen-binding molecule in said contacted sample; and
relating said measured complex concentration to the concentration of antigen-binding molecule in said sample to thereby determine the amount or level of said polypeptide in said sample.

36. The method of claim 35, wherein the concentration of said antigen-binding molecule in said biological sample is compared to a reference level of said antigen-binding molecule corresponding to said lytic phase.

37. The method of claim 16, further comprising
contacting the biological sample with an antigen corresponding to at least a portion of a polypeptide encoded by said gene;
measuring the level of antigen-specific T cell proliferation in said contacted sample to thereby determine the amount or level of said polypeptide in said sample.

38. The method of claim 37, wherein the level of said antigen-specific T cell proliferation in said biological sample is compared to a reference level of antigen-specific T cell proliferation corresponding to said lytic phase.

39. A method for the treatment or prophylaxis of chronic chlamydial infection in a patient, the method comprising administering to said patient an agent that modulates the expression of a gene or the level or functional activity of an expression product of said gene, wherein said gene is selected from pyk, nlpD, Cpn0585, or a gene belonging to the same regulatory or biosynthetic pathway as pyk, nlpD or Cpn0585, and that has been identified by a screening assay comprising:
contacting a preparation comprising a polypeptide encoded by said gene, or biologically active fragment of said polypeptide, or a genetic sequence that modulates the expression of said gene, with said agent; and
detecting a change in the level or functional activity of said polypeptide or biologically active fragment thereof, or of a product expressed from said genetic sequence.

40. A method for treatment or prophylaxis of a chronic chlamydial infection in a patient, said method comprising administering to said patient an effective amount of an agent that modulates the expression of a gene or the level or functional activity of an expression product of said gene, wherein said gene is selected from pyk, nlpD, Cpn0585, or a gene belonging to the same regulatory or biosynthetic pathway as pyk, nlpD, for a time, wherein said agent is formulated with a pharmaceutically acceptable carrier or diluent.

41. A method for treatment or prophylaxis of a chronic infection caused by an organism of the Chlamydiaceae family in a patient, said method comprising administering to said patient an effective amount of an agent that modulates the expression of a gene or the level or functional activity of an expression product of said gene, wherein said gene is selected from pyk, nlpD, Cpn0585, or a gene belonging to the same regulatory or biosynthetic pathway as pyk, nlpD, for a time and under conditions sufficient to treat or prevent said infection.

42. A method for treatment or prophylaxis of a lytic or chronic infection caused by an organism of the Chlamydiaceae family in a patient, said method comprising sequentially or simultaneously administering to said patient effective amounts of a first agent and a second agent for a time and under conditions sufficient to treat or prevent said infection, wherein said first agent modulates the expression of a first gene that is expressed in the persistent phase of the developmental cycle of said organism at a higher level than in the lytic phase of said developmental cycle or modulates the level or functional activity of an expression product of said first gene, and wherein said second agent modulates the expression of a second gene that is expressed at a higher level in said lytic phase than in said persistent phase or modulates the level or functional activity of an expression product of said second gene.

43. A method for treatment or prophylaxis of a lytic or chronic infection caused by an organism of the Chlamydiaceae family in a patient, said method comprising sequentially or simultaneously administering to said patient an effective amount of a first agent that modulates the expression of a first gene that is expressed at a higher level in the persistent phase of the developmental cycle of said organism than in the lytic phase of said developmental cycle or that modulates the level or functional activity of an expression product of said first gene, for a time and under conditions sufficient to cause said organism to enter said lytic phase, together with an effective amount of a second agent that modulates the expression of a second gene that is expressed at a higher level in said lytic phase than in said persistent phase or that modulates the level or functional activity of an expression product of said second gene, for a time and under conditions sufficient to kill, attenuate or otherwise inactivate said organism, wherein said first gene is selected from pyk, nlpD or Cpn0585, or a gene belonging to the same regulatory or biosynthetic pathway as pyk, nlpD or Cpn0585.

44. A method for treatment or prophylaxis of a lytic or chronic infection caused by an organism of the Chlamydiaceae family in a patient, said method comprising sequentially or simultaneously administering to said patient effective amounts of a first immunopotentiating agent and a second immunopotentiating agent for a time and under conditions sufficient to treat or prevent said infection, said first immunopotentiating agent being selected from a first proteinaceous molecule comprising at least a portion of a polypeptide that is expressed at a higher level in the persistent phase of the developmental cycle of said organism than in the lytic phase of said developmental cycle, or a polynucleotide from which said first proteinaceous molecule is expressible, said second immunopotentiating agent being selected from a second proteinaceous molecule comprising at least a portion of a polypeptide that is expressed at a higher level in said lytic phase than in said persistent phase, or a polynucleotide from which said second proteinaceous molecule is expressible, wherein the polypeptide of said first proteinaceous molecule is selected from Pyk, NlpD or CPn0585.

45. The method of claim 44, wherein the polypeptide of said second proteinaceous molecule is MOMP.

46. A method for treatment or prophylaxis of a lytic or chronic infection caused by an organism of the Chlamydiaceae family in a patient, said method comprising sequentially or simultaneously administering to said patient effective amounts of a first antigen that is expressed at a higher level in the persistent phase of the developmental cycle of said organism than in the lytic phase of said developmental cycle, and a second antigen that is expressed at a higher level in said lytic phase than in said persistent phase, wherein the first antigen comprises at least a portion of a polypeptide selected from Pyk, NlpD or CPn0585.

47. The method of claim 46, wherein the second antigen comprises at least a portion of MOMP.

48. A method for treatment or prophylaxis of a chronic chlamydial infection in a patient, said method comprising administering to said patient an antigen that is expressed at a higher level in the persistent phase of the developmental cycle of an organism of the Chlamydiaceae family than in the lytic phase of said developmental cycle, wherein said antigen comprises at least a portion of a polypeptide selected from Pyk, NlpD or CPn0585, and is formulated with a pharmaceutically acceptable carrier or diluent.

49. A method for treatment or prophylaxis of a chronic chlamydial infection in a patient, said method comprising administering to said patient a first antigen that is expressed at a higher level in the persistent phase of the developmental cycle of an organism of the Chlamydiaceae family than in the lytic phase of said developmental cycle, together with a second antigen that is expressed at a higher level in said lytic phase than in said persistent phase, wherein the first antigen comprises at least a portion of a polypeptide selected from Pyk, NlpD or CPn0585, and wherein said antigens are formulated with a pharmaceutically acceptable carrier or diluent.

50. The method of claim 49, wherein the second antigen comprises at least a portion of MOMP.

51. A method for treatment or prophylaxis of a chronic chlamydial infection in a patient, said method comprising administering to said patient at least one antigen that is expressed at a higher level in the persistent phase of the developmental cycle of an organism of the Chlamydiaceae family than in the lytic phase of said developmental cycle, wherein the or each antigen is selected from Pyk, NlpD or CPn0585, or a biologically active fragment thereof.

52. A method for treatment or prophylaxis of a chronic chlamydial infection in a patient, said method comprising administering to said patient at least one antigen that is expressed at a higher level in the persistent phase of the developmental cycle of an organism of the Chlamydiaceae family than in the lytic phase of said developmental cycle and that is selected from Pyk, NlpD or CPn0585, or a biologically active fragment thereof, together with at least one antigen that is expressed at a higher level in said lytic phase than in said persistent phase.


SEQUENCE LISTING (66 sequences)

SEQ ID NO: 1 - DNA, 2019 bp, Chlamydia pneumoniae
  CDS (1)..(2019)
atatcattaagacgtaaaatccttaggccaaataatttttcaattggagattgtagtagcaacatggcaacacccgctcaaaaatcccctacatttcaagatcctagttttgtaagagagctaggcagtaaccaccctgtcttttccccgctaacgcttgaggaaagaggggagatggcaatagctcgagtccagcagtgtggatggaatcatacaattgttaaggtaagtcttattattcttgctcttcttactattttagggggaggattactcgtaggattgctgccagcagttcctatgtttattggaacaggtctgattgctttgggagccgttatatttgctttggctttgattttatgtctttatgattctcagggccttcctgaggaactccctccggttcctgaaccacaacaaattcagattgaagatttaagaaacgagaccagagaagttcttgaagggactcttttagaggttctcttaaaggatagagacgctaaggaccctgcggtgccccaggtggttgtagactgtgaaaagcgtcttggaatgttggatcgtaagctgcgacgtgaagaggagattctgtatcgctcgacggcccatcttaaagacgaggaaaggtatgagttcttgctggagctcttggaaatgcgtagtctggttgccgatcggctagaatttaaccgtagaagttatgagcgatttgttcaaggaattatgacagttagatcagaggagggggaaaaagagatttctcgtctacaagatctaatcagtttgcagcagcagacggtgcaagatttaaggagtcggatcgatgacgagcagaagagatgctggacggctttacaacgtattaaccaatctcagaaggatatacaacgggctcatgatcgcgaggcttcgcagcgtgcctgtgagggcacagagatggattgtgcagaacgccagcaactggagaaggatttaaggagacagctgaaatctatgcaggagtggattgagatgaggggcacaatccatcaacaagagaaggcttggcgtaagcagaatgccaaattagaaagattacaagaggatctgagacttactgggattgcttttgacgaacaatctctgttctatcgcgaatataaagagaaatatctgagtcagaaactagatatgcaaaagattttacaggaagtcaacgcagagaaaagtgagaaggcttgcttagagagtctggtccatgactatgagaagcagctcgaacaaaaagatgctaatctgaagaaagcagcagctgtttgggaagaagaattagggaagcagcaacaggaagactacgaacaaacccaagaaattagacgtctgagtacattcattcttgagtaccaggacagtctgcgtgaggcagaaaaagttgagaaagatttccaagagctacaacaaaggtatagccgtcttcaagaggagaaacaggtaaaagaaaaaatcttagaagaaagtatgaatcattttgccgatctctttgagaaggctcaaaaggaaaacatggcctacaagaagaagttagcggatttagagggtgccgctgctcctactgagatcggtgaggacgatgactgggtactcacagattctgcttctctcagccagaagaagatccgcgaactcgtggaagagaatcaagaactcctgaaagcacttgcatttaaatctaacgaattgactcaactggttgccgatgctgtagaagctgaaaaagaaatcagcaagcttcgagaacacatagaagagcagaaagaaggattacgagctcttgataagatgcatgcacaagcgatcaaagattgcgaagctgctcagagaaaatgctgtgaccttgagagccttctctctcctgttcgagaagatgctggaatgagatttgagctagaggtcgagcttcaaagattgcaagaagaaaatgcacagcttagagcggaggttgaaagactagagcaagagcaatttcaaggataa

SEQ ID NO: 2 - protein, 672 aa, Chlamydia pneumoniae
ISLRRKILRPNNFSIGDCSSNMATPAQKSPTFQDPSFVRELGSNHPVFSPLTLEERGEMAIARVQQCGWNHTIVKVSLIILALLTILGGGLLVGLLPAVPMFIGTGLIALGAVIFALALILCLYDSQGLPEELPPVPEPQQIQIEDLRNETREVLEGTLLEVLLKDRDAKDPAVPQVVVDCEKRLGMLDRKLRREEEILYRSTAHLKDEERYEFLLELLEMRSLVADRLEFNRRSYERFVQGIMTVRSEEGEKEISRLQDLISLQQQTVQDLRSRIDDEQKRCWTALQRINQSQKDIQRAHDREASQRACEGTEMDCAERQQLEKDLRRQLKSMQEWIEMRGTIHQQEKAWRKQNAKLERLQEDLRLTGIAFDEQSLFYREYKEKYLSQKLDMQKILQEVNAEKSEKACLESLVHDYEKQLEQKDANLKKAAAVWEEELGKQQQEDYEQTQEIRRLSTFILEYQDSLREAEKVEKDFQELQQRYSRLQEEKQVKEKILEESMNHFADLFEKAQKENMAYKKKLADLEGAAAPTEIGEDDDWVLTDSASLSQKKIRELVEENQELLKALAFKSNELTQLVADAVEAEKEISKLREHIEEQKEGLRALDKMHAQAIKDCEAAQRKCCDLESLLSPVREDAGMRFELEVELQRLQEENAQLRAEVERLEQEQFQG

SEQ ID NO: 3 - DNA, 738 bp, Chlamydia pneumoniae
  CDS (1)..(738)
gctgttgatcagagaaatgcaggcagtgaggtaaatatgaatcgtagagacatggtaataacagctgtcgtagtgaatgctatattgcttgtggctcttttcgtcacatcaaagcgtattggcgtcaaggactatgacgagggattccgtaattttgcttctagcaaggttacacaagcagtagtttcagaagaaaaagtcatagaaaagcctgtagtcgcagaagtgcctagccgtcctatcgctaaagagactctagctgcacagtttattgaaagtaagccggttattgtaaccacaccacccgtgcctgttgttagcgaaaccccagaagtgcctactgtggcagttccgcctcagcctgttcgtgagacagtaaaagaggaacaagctccttatgctactgttgtagtgaaaaaaggagattttctcgaacgcattgcgagagcaaatcatactaccgttgcaaaattgatgcagatcaatgatcttaccaccacccaacttaaaattggtcaggtcatcaaagtccctacgtctcaagatgtcagcaacgaaaaaactcctcaaacacagaccgcaaaccctgaaaattattatatcgtccaagaaggggatagcccgtggacaatagcattgcgtaaccatattcgattggatgatttgctaaaaatgaatgatctcgatgaatataaagcccggcgccttaagcctggagatcagttgcgcatacgttga

SEQ ID NO: 4 - protein, 245 aa, Chlamydia pneumoniae
AVDQRNAGSEVNMNRRDMVITAVVVNAILLVALFVTSKRIGVKDYDEGFRNFASSKVTQAVVSEEKVIEKPVVAEVPSRPIAKETLAAQFIESKPVIVTTPPVPVVSETPEVPTVAVPPQPVRETVKEEQAPYATVVVKKGDFLERIARANHTTVAKLMQINDLTTTQLKIGQVIKVPTSQDVSNEKTPQTQTANPENYYIVQEGDSPWTIALRNHIRLDDLLKMNDLDEYKARRLKPGDQLRIR

SEQ ID NO: 5 - DNA, 1185 bp, Chlamydia pneumoniae
  CDS (1)..(1185)
tctttagaggtgagtatgaaaaaactcttaaagtcggcgttattatccgccgcatttgctggttctgttggctccttacaagccttgcctgtagggaacccttctgatccaagcttattaattgatggtacaatatgggaaggtgctgcaggagatccttgcgatccttgcgctacttggtgcgacgctattagcttacgtgctggattttacggagactatgttttcgaccgtatcttaaaagtagatgcacctaaaacattttctatgggagccaagcctactggatccgctgctgcaaactatactactgccgtagatagacctaacccggcctacaataagcatttacacgatgcagagtggttcactaatgcaggcttcattgccttaaacatttgggatcgctttgatgttttctgtactttaggagcttctaatggttacattagaggaaactctacagcgttcaatctcgttggtttattcggagttaaaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaacccaagggctataaaggcgttgctttccccttgccaacagacgctggcgtagcaacagctactggaacaaagtctgcgaccatcaattatcatgaatggcaagtaggagcctctctatcttacagactaaactctttagtgccatacattggagtacaatggtctcgagcaacttttgatgctgataacatccgcattgctcagccaaaactacctacagctgttttaaacttaactgcatggaacccttctttactaggaaatgccacagcattgtctactactgattcgttctcagacttcatgcaaattgtttcctgtcagatcaacaagtttaaatctagaaaagcttgtggagttactgtaggagctactttagttgatgctgataaatggtcacttactgcagaagctcgtttaattaacgagagagctgctcacgtatctggtcagttcagattctaa

SEQ ID NO: 6 - protein, 394 aa, Chlamydia pneumoniae
SLEVSMKKLLKSALLSAAFAGSVGSLQALPVGNPSDPSLLIDGTIWEGAAGDPCDPCATWCDAISLRAGFYGDYVFDRILKVDAPKTFSMGAKPTGSAAANYTTAVDRPNPAYNKHLHDAEWFTNAGFIALNIWDRFDVFCTLGASNGYIRGNSTAFNLVGLFGVKGTTVNANELPNVSLSNGVVELYTDTSFSWSVGARGALWECGCATLGAEFQYAQSKPKVEELNVICNVSQFSVNKPKGYKGVAFPLPTDAGVATATGTKSATINYHEWQVGASLSYRLNSLVPYIGVQWSRATFDADNIRIAQPKLPTAVLNLTAWNPSLLGNATALSTTDSFSDFMQIVSCQINKFKSRKACGVTVGATLVDADKWSLTAEARLINERAAHVSGQFRF

SEQ ID NO: 7 - DNA, 1047 bp, Chlamydia pneumoniae
  CDS (1)..(1047)
ggcccctttgacatgaatagcaagatgctaaaacatttacgtttagcaaccctttccttctctatgttcttcgggattgtatcttctcccgcagtatatgccctaggggctggaaaccctgcagctccagtactcccaggtgtgaatcctgagcaaacgggatggtgtgccttccaactttgtaatagttacgatctttttgctgctcttgcaggaagcctcaaatttgggttctatggagattatgtcttctcagaaagtgcccatattaccaatgtccctgtcattacctccgttacgacttcaggcacaggaacaacgccaaccattacctctacaactaaaaacgtagactttgatcttaacaacagctccatcagctcgagctgtgtttttgcaaccatagctctacaggaaacatccccagctgccattccccttttagatatagccttcactgcacgtgtcggaggacttaagcagtactaccgcctccctctcaatgcttacagagacttcacttcaaatcctttaaatgcagaatctgaagttacagatggtctcattgaagtccagtcagactatggaattgtctggggtctgagtttacaaaaagtattgtggaaagatggagtgtcttttgtaggggtgagcgctgactaccgtcacggttccagtcccatcaactatatcatcgtttacaacaaggccaaccccgagatctatttcgatgctactgatggaaacctaagctataaagaatggtctgcaagcatcggcatctctacgtatcttaatgactatgtgcttccctatgcatccgtatctataggaaatacttcaagaaaagctccttctgatagcttcacagaactcgaaaagcaatttacgaattttaaatttaaaattcgtaaaatcacaaacttcgacagagtaaacttctgcttcggaactacctgctgcatctcaaataacttctactatagtgtagaaggccgttggggatatcagcgtgctatcaacattacgtcaggtctgcagttttag

SEQ ID NO: 8 - protein, 348 aa, Chlamydia pneumoniae
GPFDMNSKMLKHLRLATLSFSMFFGIVSSPAVYALGAGNPAAPVLPGVNPEQTGWCAFQLCNSYDLFAALAGSLKFGFYGDYVFSESAHITNVPVITSVTTSGTGTTPTITSTTKNVDFDLNNSSISSSCVFATIALQETSPAAIPLLDIAFTARVGGLKQYYRLPLNAYRDFTSNPLNAESEVTDGLIEVQSDYGIVWGLSLQKVLWKDGVSFVGVSADYRHGSSPINYIIVYNKANPEIYFDATDGNLSYKEWSASIGISTYLNDYVLPYASVSIGNTSRKAPSDSFTELEKQFTNFKFKIRKITNFDRVNFCFGTTCCISNNFYYSVEGRWGYQRAINITSGLQF

SEQ ID NO: 9 - DNA, 1461 bp, Chlamydia pneumoniae
  CDS (1)..(1461)
gacagtatgatcacacgcactaaaattatttgcactatagggccagcaacgaatagtccagagatgttagcaaaacttctagatgctgggatgaacgtagcaagattaaatttcagtcatgggagtcacgaaactcatggacaggctattggatttctcaaggagttaagggagcagaagcgggttcctttagcaattatgctagatactaaggggcctgaaattcgtttagggaatattcctcagccaatttcggtttctcagggacaaaagcttcgtctggtaagtagtgatatcgatgggagtgctgaagggggagtgtctctctatcctaaggggatatttccctttgttcctgagggtgctgatgttttaatagatgatggctacattcatgctgttgttgtctcttcagaggctgattctttagaattagagtttatgaacagtggccttctcaagtctcataaatctttgagtatccgaggtgttgatgttgctcttccctttatgacagagaaagatattgcggatcttaagtttggggtagagcagaatatggatgtggttgctgcatcttttgtgcgctacggtgaagatattgaaactatgcgcaagtgtttagcagacttaggcaatcctaagatgcccatcattgcaaaaatagaaaatcgtttaggggtagaaaatttctctaagattgccaagcttgcggatggaattatgattgctagaggagatttaggaatcgagctttctgtcgttgaagtcccaaatttgcaaaagatgatggctaaggtttctagagaaacaggtcacttctgtgtgactgcaacgcagatgctagaatctatgattcgcaatgtcttacctacacgagctgaagtctctgatattgccaatgcaatttatgatggttcttcagcagtgatgttgtcaggggaaactgcatctggagcccatcccgtggctgccgtgaaaatcatgcgttctgtgattttagaaacagaaaagaatctctcccatgattcattcttaaaattagacgatagcaatagcgctcttcaggtgtccccctatctctcagccattggattggcaggcattcagattgcagaaagggcagacgccaaagctcttattgtttatacagaatcaggaagttctccgatgtttctctctaaatatcgtccgaaattccctatcattgccgtgactccaagcacttctgtttactatcgcctagctttggaatggggggtctatcctatgcttacccaggaaagtgatcgcgctgtatggagacatcaggcctgtatttatggcatagaacagggcattctctctaattatgatcggattcttgtgcttagcagaggagcctgtatggaagaaacaaataatcttaccctgacaatagtgaatgatattttgactgggtcggaatttcctgaaacctag

SEQ ID NO: 10 - protein, 486 aa, Chlamydia pneumoniae
DSMITRTKIICTIGPATNSPEMLAKLLDAGMNVARLNFSHGSHETHGQAIGFLKELREQKRVPLAIMLDTKGPEIRLGNIPQPISVSQGQKLRLVSSDIDGSAEGGVSLYPKGIFPFVPEGADVLIDDGYIHAVVVSSEADSLELEFMNSGLLKSHKSLSIRGVDVALPFMTEKDIADLKFGVEQNMDVVAASFVRYGEDIETMRKCLADLGNPKMPIIAKIENRLGVENFSKIAKLADGIMIARGDLGIELSVVEVPNLQKMMAKVSRETGHFCVTATQMLESMIRNVLPTRAEVSDIANAIYDGSSAVMLSGETASGAHPVAAVKIMRSVILETEKNLSHDSFLKLDDSNSALQVSPYLSAIGLAGIQIAERADAKALIVYTESGSSPMFLSKYRPKFPIIAVTPSTSVYYRLALEWGVYPMLTQESDRAVWRHQACIYGIEQGILSNYDRILVLSRGACMEETNNLTLTIVNDILTGSEFPET

SEQ ID NO: 11 - DNA, 1665 bp, Chlamydia trachomatis
  CDS (1)..(1665)
tctatgcgaataggagatcctatgaacaaactcatcagacgagcagtgacgatcttcgcggtgactagtgtggcgagtttatttgctagcggggtgttagagacctctatggcagagtctctctctacaaacgttattagcttagctgacaccaaagcgaaagacaacacttctcataaaagcaaaaaagcaagaaaaaaccacagcaaagagactcccgtagaccgtaaagaggttgctccggttcatgagtctaaagctacaggacctaaacaggattcttgctttggcagaatgtatacagtcaaagttaatgatgatcgcaatgttgaaatcacacaagctgttcctgaatatgctacggtaggatctccctatcctattgaaattactgctacaggtaaaagggattgtgttgatgttatcattactcagcaattaccatgtgaagcagagttcgtacgcagtgatccagcgacaactcctactgctgatggtaagctagtttggaaaattgaccgcttaggacaaggcgaaaagagtaaaattactgtatgggtaaaacctcttaaagaaggttgctgctttacagctgcaacagtatgcgcttgtccagagatccgttcggttacaaaatgtggacaacctgctatctgtgttaaacaagaaggcccagagaatgcttgtttgcgttgcccagtagtttacaaaattaatatagtgaaccaaggaacagcaacagctcgtaacgttgttgttgaaaatcctgttccagatggttacgctcattcttctggacagcgtgtactgacgtttactcttggagatatgcaacctggagagcacagaacaattactgtagagttttgtccgcttaaacgtggtcgtgctaccaatatagcaacggtttcttactgtggaggacataaaaatacagcaagcgtaacaactgtgatcaacgagccttgcgtacaagtaagtattgcaggagcagattggtcttatgtttgtaagcctgtagaatatgtgatctccgtttccaatcctggagatcttgtgttgcgagatgtcgtcgttgaagacactctttctcccggagtcacagttcttgaagctgcaggagctcaaatttcttgtaataaagtagtttggactgtgaaagaactgaatcctggagagtctctacagtataaagttctagtaagagcacaaactcctggacaattcacaaataatgttgttgtgaagagctgctctgactgtggtacttgtacttcttgcgcagaagcgacaacttactggaaaggagttgctgctactcatatgtgcgtagtagatacttgtgaccctgtttgtgtaggagaaaatactgtttaccgtatttgtgtcaccaacagaggttctgcagaagatacaaatgtttctttaatgcttaaattctctaaagaactgcaacctgtatccttctctggaccaactaaaggaacgattacaggcaatacagtagtattcgattcgttacctagattaggttctaaagaaactgtagagttttctgtaacattgaaagcagtatcagctggagatgctcgtggggaagcgattctttcttccgatacattgactgttccagtttctgatacagagaatacacacatctattaa

SEQ ID NO: 12 - protein, 554 aa, Chlamydia trachomatis
SMRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 13 - DNA, 1203 bp, Chlamydia trachomatis
  CDS (1)..(1203)
ttatacaatttagaggtaagaatgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcgccacttggtgtgacgctatcagcatgcgtgttggttactacggagactttgttttcgaccgtgttttgaaaactgatgtgaataaagaatttcagatgggtgccaagcctacaactgatacaggcaatagtgcagctccatccactcttacagcaagagagaatcctgcttacggccgacatatgcaggatgctgagatgtttacaaatgccgcttgcatggcattgaatatttgggatcgttttgatgtattctgtacattaggagccaccagtggatatcttaaaggaaactctgcttctttcaatttagttggattgtttggagataatgaaaatcaaaaaacggtcaaagcggagtctgtaccaaatatgagctttgatcaatctgttgttgagttgtatacagatactacttttgcgtggagcgtcggcgctcgcgcagctttgtgggaatgtggatgtgcaactttaggagcttcattccaatatgctcaatctaaacctaaagtagaagaattaaacgttctctgcaatgcagcagagtttactattaataaacctaaagggtatgtaggtaaggagtttcctcttgatcttacagcaggaacagatgctgcgacaggaactaaggatgcctctattgattaccatgaatggcaagcaagtttagctctctcttacagactgaatatgttcactccctacattggagttaaatggtctcgagcaagctttgatgccgatacgattcgtatagcccagccaaaatcagctacagctatttttgatactaccacgcttaacccaactattgctggagctggcgatgtgaaaactggcgcagagggtcagctcggagacacaatgcaaatcgtttccttgcaattgaacaagatgaaatctagaaaatcttgcggtattgcagtaggaacaactattgtggatgcagacaaatacgcagttacagttgagactcgcttgatcgatgagagagcagctcacgtaaatgcacaattccgcttctaa

SEQ ID NO: 14 - protein, 400 aa, Chlamydia trachomatis
LYNLEVRMKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 15 - DNA, 768 bp, Chlamydia trachomatis
  CDS (1)..(768)
gcagttggttgggatggaagaggttcttttatgaatcgtagaaacacgatgattgtagcaactgctgtgaatgcagtgctattggcagtgctgtttatgaccgcgcgccattcagagcaagaaatagagtattctcagaaaatagctcctattaaaatcttagagcccgttccggttgttgataaggctccagagaagttagagaaaaagcctgaggtgattgcgaagccttctcaggtcgttagaaatcctgtcgtttctaaagctgaacttgctgcgcaatttgcagacaaaaatcctaagacagagaaggaatctagcgggggctctaaagagatttcatctacccctgtagaatcgacgactcctgtcgctccagaaatttctgttgtgaacgctaaggtagtagagaaaactcctgaaaaagaggaattctctactgttattgttaagaaaggagactttttagaacgtatagctagatccaatcacactacagtttctgcattgatgcagttgaatgacttatcttcgacacagttacagataggacaagtgttacgagttcctaaaacgaataagacagagaaggatcttcaagtgaagactccaaatctggaagattactatgtagtcaaggaaggagatagtccttgggccattgcattgagtaatggtattcgtttggatgagctgttgaagttaaatggattagatgagcagaaagctcgtagattacgtccaggggatagattacgaattcgataa

SEQ ID NO: 16 - protein, 255 aa, Chlamydia trachomatis
AVGWDGRGSFMNRRNTMIVATAVNAVLLAVLFMTARHSEQEIEYSQKIAPIKILEPVPVVDKAPEKLEKKPEVIAKPSQVVRNPVVSKAELAAQFADKNPKTEKESSGGSKEISSTPVESTTPVAPEISVVNAKVVEKTPEKEEFSTVIVKKGDFLERIARSNHTTVSALMQLNDLSSTQLQIGQVLRVPKTNKTEKDLQVKTPNLEDYYVVKEGDSPWAIALSNGIRLDELLKLNGLDEQKARRLRPGDRLRIR

SEQ ID NO: 17 - DNA, 1494 bp, Chlamydia trachomatis
  CDS (1)..(1494)
aagatattctactcactaataccggtatcccgatttatgatcgctagaacgaaaattatttgtacgataggccctgcaacgaatacccctgagatgctggaaaagcttcttgatgcagggatgaatgtagctcgccttaattttagccacgggacccatgaaagccatggccggaccatcgctattcttaaagaactacgagagaagcgccaagttcctttagctattatgctagatacaaaaggtcccgaaattcgtttaggccaagtagaatctcctataaaagtacagcctggggatcgtcttactctcgttagcaaagaaattttaggatccaaagaaagcggcgttactctttatccaagttgtgtattcccttatgttagagaacgagctcctgttctcattgatgatgggtatatccaagcagtggtggtcaatgctcaagagcatatggtggaaatagagtttcaaaattcaggagaaataaaatccaacaaatctcttagcatcaaagatatcgatgttgctcttcctttcatgacagagaaggatattgcagacttaaaatttggggtagaacaagaactcgatcttatcgctgcttcgttcgtcagatgtaatgaagatattgacagcatgcgtaaagttttggaaagctttggtcgtcctaatatgcccatcattgctaaaatagaaaatcatttaggagtacaaaatttccaagagatcgctagagctgctgatggtatcatgattgcacgcggggatcttggtattgaattgtctattgttgaagttcctggactacaaaaatttatggcccgagcatcgagggaaacgggtcggttttgtatcactgcaacgcaaatgctcgagtcaatgattcgcaacccccttcctacacgagccgaagtctctgacgttgccaacgccatttacgatggaacctctgcagtcatgttgtctggagaaactgccttaggagcccatcctgtacatgcagtaaaaacaatgcgttccattatccaagagactgagaagactttcgattaccacgcttttttccagctgaacgacaaaaacagcgctctcaaagtttctccttatcttgaagccattgggttttctggaatccaaattgcagaaaaagcatctgccaaagccattattgtgtatacccagacgggaggatctccgatgtttttatccaaatatcgaccttatctccctattattgctgttacccctaaccgcaatgtgtactatcgtttagctgtagaatggggagtatatcctatgctaaccctagaatcgaaccgtacagtctggcgtcaccaagcttgtgtatatggagtagaaaaaggaattctttctaactatgataaaattcttgtcttcagccgcggagctgggatgcaagacaccaacaatctcaccttgacaactgtgcatgatgtgctatccccctctcttgacgagatagttccataa

SEQ ID NO: 18 - protein, 497 aa, Chlamydia trachomatis
KIFYSLIPVSRFMIARTKIICTIGPATNTPEMLEKLLDAGMNVARLNFSHGTHESHGRTIAILKELREKRQVPLAIMLDTKGPEIRLGQVESPIKVQPGDRLTLVSKEILGSKESGVTLYPSCVFPYVRERAPVLIDDGYIQAVVVNAQEHMVEIEFQNSGEIKSNKSLSIKDIDVALPFMTEKDIADLKFGVEQELDLIAASFVRCNEDIDSMRKVLESFGRPNMPIIAKIENHLGVQNFQEIARAADGIMIARGDLGIELSIVEVPGLQKFMARASRETGRFCITATQMLESMIRNPLPTRAEVSDVANAIYDGTSAVMLSGETALGAHPVHAVKTMRSIIQETEKTFDYHAFFQLNDKNSALKVSPYLEAIGFSGIQIAEKASAKAIIVYTQTGGSPMFLSKYRPYLPIIAVTPNRNVYYRLAVEWGVYPMLTLESNRTVWRHQACVYGVEKGILSNYDKILVFSRGAGMQDTNNLTLTTVHDVLSPSLDEIVP

SEQ ID NO: 19 - DNA, 1161 bp, Chlamydia trachomatis
  CDS (1)..(1161)
  misc_feature (305)..(305): The ′Xaa′ at location 305 stands for Glu.
atgaaaaaactcttgaaatcggtattagcatttgccgttttgggttctgcttcctccttgcatgctctgcctgtggggaatcctgctgaaccaagccttatgattgacgggattctttgggaaggtttcggtggagatccttgcgatccttgcacaacttggtgtgatgccatcagcctacgtctcggctactatggggacttcgtttttgatcgtgttttgaaaacagacgtgaacaaacagttcgaaatgggagcagctcctacaggagatgcagaccttactacagcacctactcctgcatcaagagagaatcccgcttatggcaagcatatgcaagatgcagaaatgttcactaatgctgcgtacatggctttaaacatttgggaccgtttcgatgtattttgtacattgggagcaactagcggatatcttaaaggtaattctgccgcctttaacttagttggtctgtttggaagagatgaaactgcagttgcagctgacgacatacctaacgtcagcttgtctcaagctgttgtcgaactctacacagacacagctttcgcttggagcgtcggtgctagagcagctttatgggagtgcggatgtgcaactttaggagcttccttccaatatgctcaatctaagccaaaagtagaggaattaaacgttctctgtaatgcggcagaattcactattaacaagcctaaaggatacgttggacaagagtttcctcttaacattaaagctggaacagttagcgctacagatactaaagatgcttccatcgattaccatgagtggcaagcaagcttggctttgtcttacagactgaatatgttcactccttacattggagttaagtggtctagagcaagctttgatgccgacactatccgcattgcgcagcctkagcttgagacctctatcttaakaatgaccacttggaacccaacgatctctggatctggtatagacgttgatacaaaaatcacggatacattacaaattgtttccttgcagctcaacaagatgaaatccagaaaatcttgcggtcttgcaattggaacaacaattgtagatgctgataaatatgcagttactgttgagacacgcttgatcgatgaaagagcagctcacgtaaatgctcagttccgtttc

SEQ ID NO: 20 - protein, 387 aa, Chlamydia trachomatis
  misc_feature (305)..(305): The ′Xaa′ at location 305 stands for Glu.
MKKLLKSVLAFAVLGSASSLHALPVGNPAEPSLMIDGILWEGFGGDPCDPCTTWCDAISLRLGYYGDFVFDRVLKTDVNKQFEMGAAPTGDADLTTAPTPASRENPAYGKHMQDAEMFTNAAYMALNIWDRFDVFCTLGATSGYLKGNSAAFNLVGLFGRDETAVAADDIPNVSLSQAVVELYTDTAFAWSVGARAALWECGCATLGASFQYAQSKPKVEELNVLCNAAEFTINKPKGYVGQEFPLNIKAGTVSATDTKDASIDYHEWQASLALSYRLNMFTPYIGVKWSRASFDADTIRIAQPXLETSILXMTTWNPTISGSGIDVDTKITDTLQIVSLQLNKMKSRKSCGLAIGTTIVDADKYAVTVETRLIDERAAHVNAQFRF

SEQ ID NO: 21 - DNA, 1443 bp, Chlamydia trachomatis
  CDS (1)..(1443)
atgatcgctagaacaaaaattatttgtacaataggccctgcaacaaacaccccagaaatgcttgaaaaacttcttgatgcggggatgaatgtagcgcgtcttaacttcagtcatggtacccacgaaagccacggccggaccattgctattcttaaggaactacgcgaaaagcgccaagtccctttagctattatgttggatacaaaaggaccagaaattcgtttaggccaagtagaatctcctataaaagtgaagccaggagaccgtctcactttaaccagtaaagaaattttgggatccaaagaagctggagtcactctttatcctagctgcgtgttccctttcgttcgcgaacgcgctcccgtcctgattgatgatggatatatccaagccgtagttgtcaatgctcaagagcatctcattgagatagaatttcagaattcaggagaaatcaagtctaataaatcacttagcatcaaagatatagacgtagccctccccttcatgacagagaaggatatcacggatctaaaattcggggtcgaacaagaacttgaccttatcgcagcatcttttgtccgatgtaacgaagacatcgatagcatgcgtaaagttttagaaaacttcggccggccaaatatgccgatcattgccaaaatagaaaatcatttaggggtacaaaatttccaagaaatagccaaagcttctgatggaattatgatcgcacgaggagatctcggcatcgaattatctatcgttgaagtccctgccttacaaaaatttatggctcgtgtgtccagagaaacaggccgtttttgtatcaccgcaacacaaatgctcgagtcaatgattcgcaatccccttcctacacgagccgaagtttccgatgtagctaatgctatccacgatggaacttccgctgtgatgttatcaggagaaactgcttcaggaacttatcctatagaagctgtaaaaactatgcgctcgatcatccaagaaacggaaaaatcctttgattaccaagcctttttccaactcaatgacaaaaatagcgctctcaaagtctctccttatcttgaagcaataggcgcttcagggatccaaatcgctgagaaagcttctgctaaagcgattattgtatacacccaaactgggggatctcccatgtttctttctaaatatcgtccctatctccccattattgccgttaccccaaaccgcaatgtatactatcgcttagcagtagaatggggcgtataccctatgctaacctcagaatctaaccgaacagtttggcgccaccaagcttgtgtctatggagtagagaaaggaatcctttcaaactatgataaaattcttgtttttagccgaggagcagggatgcaggacacgaataaccttactctgactactgtaaacgatgttttatctccttctcttgaa

SEQ ID NO: 22 - protein, 481 aa, Chlamydia trachomatis
MIARTKIICTIGPATNTPEMLEKLLDAGMNVARLNFSHGTHESHGRTIAILKELREKRQVPLAIMLDTKGPEIRLGQVESPIKVKPGDRLTLTSKEILGSKEAGVTLYPSCVFPFVRERAPVLIDDGYIQAVVVNAQEHLIEIEFQNSGEIKSNKSLSIKDIDVALPFMTEKDITDLKFGVEQELDLIAASFVRCNEDIDSMRKVLENFGRPNMPIIAKIENHLGVQNFQEIAKASDGIMIARGDLGIELSIVEVPALQKFMARVSRETGRFCITATQMLESMIRNPLPTRAEVSDVANAIHDGTSAVMLSGETASGTYPIEAVKTMRSIIQETEKSFDYQAFFQLNDKNSALKVSPYLEAIGASGIQIAEKASAKAIIVYTQTGGSPMFLSKYRPYLPIIAVTPNRNVYYRLAVEWGVYPMLTSESNRTVWRHQACVYGVEKGILSNYDKILVFSRGAGMQDTNNLTLTTVNDVLSPSLE

SEQ ID NO: 23 - DNA, 1662 bp, Chlamydia trachomatis
  CDS (1)..(1662)
atgcgaataggagatcctatgaacaaactcatcagacgagctgtgacgatcttcgcggtgactagtgtggcgagtttatttgctagcggggtgttagagacctctatggcagagtctctctctaccaacgttattagcttagctgacaccaaagcgaaagagaccacttctcatcaaaaagacagaaaagcaagaaaaaatcatcaaaataggacttccgtagtccgtaaagaggttactgcagttcgtgatactaaagctgtagagcctagacaggattcttgctttggcaaaatgtatacagtcaaagttaatgatgatcgtaatgtagaaatcgtgcagtccgttcctgaatatgctacggtaggatctccatatcctattgagattactgctatagggaaaagagactgtgttgatgtaatcattacacagcaattaccatgcgaagcagagtttgttagcagtgatccagctactactcctactgctgatggtaagctagtttggaaaattgatcggttaggacagggcgaaaagagtaaaattactgtatgggtaaaacctcttaaagaaggttgctgctttacagctgcaacggtttgtgcttgtccagagatccgttcggttacgaaatgtggccagcctgctatctgtgttaaacaggaaggtccagaaagcgcatgtttgcgttgcccagtaacttatagaattaatgtagtcaaccaaggaacagcaacagcacgtaatgttgttgtggaaaatcctgttccagatggctatgctcatgcatccggacagcgtgtattgacatatactcttggggatatgcaacctggagaacagagaacaatcaccgtggagttttgtccgcttaaacgtggtcgagtcacaaatattgctacagtttcttactgtggtggacacaaaaatactgctagcgtaacaacagtgatcaatgagccttgcgtgcaagttaacatcgagggagcagattggtcttatgtttgtaagcctgtagaatatgttatctctgtttctaaccctggtgacttagttttacgagacgttgtaattgaagatacgctttctcctggaataactgttgttgaagcagctggagctcagatttcttgtaataaattggtttggactttgaaggaactcaatcctggagagtctttacaatataaggttctagtaagagctcaaactccagggcaattcacaaacaacgttgttgtgaaaagttgctctgattgcggtatttgtacttcttgcgcagaagcaacaacttactggaaaggagttgctgctactcatatgtgcgtagtagatacttgtgatcctatttgcgtaggagagaacactgtttatcgtatctgtgtgacaaacagaggttctgctgaagatacaaatgtgtccttaattttgaaattctctaaagaattacaacctatatctttctctggaccaactaaaggaaccattacaggaaacacggtagtgtttgattcgttacctagattaggttctaaagaaactgtagagttttctgtaacgttgaaagcagtatccgctggagatgctcgtggggaagctattctttcttccgatacattgacagttcctgtatctgatacggagaatacacatatctat

SEQ ID NO: 24 - protein, 554 aa, Chlamydia trachomatis
MRIGDPMNKLIRRAVTIFAVTSVASLFASGVLETSMAESLSTNVISLADTKAKETTSHQKDRKARKNHQNRTSVVRKEVTAVRDTKAVEPRQDSCFGKMYTVKVNDDRNVEIVQSVPEYATVGSPYPIEITAIGKRDCVDVIITQQLPCEAEFVSSDPATTPTADGKLVWKIDRLGQGEKSKITVWVKPLKEGCCFTAATVCACPEIRSVTKCGQPAICVKQEGPESACLRCPVTYRINVVNQGTATARNVVVENPVPDGYAHASGQRVLTYTLGDMQPGEQRTITVEFCPLKRGRVTNIATVSYCGGHKNTASVTTVINEPCVQVNIEGADWSYVCKPVEYVISVSNPGDLVLRDVVIEDTLSPGITVVEAAGAQISCNKLVWTLKELNPGESLQYKVLVRAQTPGQFTNNVVVKSCSDCGICTSCAEATTYWKGVAATHMCVVDTCDPICVGENTVYRICVTNRGSAEDTNVSLILKFSKELQPISFSGPTKGTITGNTVVFDSLPRLGSKETVEFSVTLKAVSAGDARGEAILSSDTLTVPVSDTENTHIY

SEQ ID NO: 25 - DNA, 729 bp, Chlamydia trachomatis
  CDS (1)..(729)
atgaatcgtagaaacacgatgattgtagcagcttctgtgaatgcagtactcttggcagtgctgtttatgacagcacgctattcagagcaagaggtagagtattcgcagaaaatagcacctattaaaattttagagccggtaccagttgttgagaaggctcctgaaaaattagaaaaaaatccagaagtgatcgctaagcccgcacaggttgtgagaaatcctgtagtctctaaagcggagcttgctgcgcaattcacagataaaaatcaaactgttgagaaagagatcaaagtctctcctaaagcgacgcctcctcctgtggttgttgaatctcctacatccgaaattcctgttgtgcaggaaaaaagtgcagacaaacctgcagagcaagaagaattttctacagttattgttaagaagggagattttttagagcgaattgctagatcccatcacactacagtttctgcattgatgcaacttaatgatttgtcgtcgacgcaattacaaataggacaagttttacgcgttcctaaaacgaataagacagagaaagatcttcaagttaagacccccaatccagaagattattatgtgattaaagaaggggatagtccttgggctatagctttgagtaatgggattcgattagatgaattgttgaaattgaatggattagatgagcagaaagctcgtaagttgcgtcctggagatagattacgaattcga

SEQ ID NO: 26 - protein, 243 aa, Chlamydia trachomatis
MNRRNTMIVAASVNAVLLAVLFMTARYSEQEVEYSQKIAPIKILEPVPVVEKAPEKLEKNPEVIAKPAQVVRNPVVSKAELAAQFTDKNQTVEKEIKVSPKATPPPVVVESPTSEIPVVQEKSADKPAEQEEFSTVIVKKGDFLERIARSHHTTVSALMQLNDLSSTQLQIGQVLRVPKTNKTEKDLQVKTPNPEDYYVIKEGDSPWAIALSNGIRLDELLKLNGLDEQKARKLRPGDRLRIR

SEQ ID NO: 27 - DNA, 1167 bp, Chlamydia pneumoniae
  CDS (1)..(1167)
atgaaaaaactcttaaagtcggcgttattatccgccgcatttgctggttctgtcggctccttacaagccttgcctgtagggaacccttctgatccaagcttattaattgatggtacaatatgggaaggtgctgcaggagatccttgcgatccttgcgctacttggtgcgacgctattagcttacgtgctggattttacggagactatgttttcgaccgtatcttaaaagtagatgcacctaaaacattttctatgggagccaagcctactggatccgctgctgcaaactatactactgccgtagatagacctaacccggcctacaataagcatttacacgatgcagagtggttcactaatgcaggcttcattgccttaaacatttgggatcgctttgatgttttctgtactttaggagcttctaatggttacattagaggaaactctacagcgttcaatctcgttggtttattcggagttaaaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaacccaagggctataaaggcgttgctttccccttgccaacagacgctggcgtagcaacagctactggaacaaagtctgcgaccatcaattatcatgaatggcaagtaggagcctctctatcttacagactaaactctttagtgccatacattggagtacaatggtctcgagcaacttttgatgctgataacatccgcattgctcagccaaaactacctacagctgttttaaacttaactgcatggaacccttctttactaggaaatgccacagcattgtctactactgattcgttctcagacttcatgcaaattgtttcctgtcagatcaacaagtttaaatctagaaaagcttgtggagttactgtaggagctactttagttgatgctgataaatggtcacttactgcagaagctcgtttaattaacgagagagctgctcacgtatctggtcagttcagattc

SEQ ID NO: 28 - protein, 389 aa, Chlamydia pneumoniae
MKKLLKSALLSAAFAGSVGSLQALPVGNPSDPSLLIDGTIWEGAAGDPCDPCATWCDAISLRAGFYGDYVFDRILKVDAPKTFSMGAKPTGSAAANYTTAVDRPNPAYNKHLHDAEWFTNAGFIALNIWDRFDVFCTLGASNGYIRGNSTAFNLVGLFGVKGTTVNANELPNVSLSNGVVELYTDTSFSWSVGARGALWECGCATLGAEFQYAQSKPKVEELNVICNVSQFSVNKPKGYKGVAFPLPTDAGVATATGTKSATINYHEWQVGASLSYRLNSLVPYIGVQWSRATFDADNIRIAQPKLPTAVLNLTAWNPSLLGNATALSTTDSFSDFMQIVSCQINKFKSRKACGVTVGATLVDADKWSLTAEARLINERAAHVSGQFRF

SEQ ID NO: 29 - DNA, 1668 bp, Chlamydia pneumoniae
  CDS (1)..(1668)
atgtccaaactcatcagacgagtagttacggtccttgcgctaacgagtatggcgagttgctttgccagcgggggtatagaggccgctgtagcagagtctctgattactaagatcgtcgctagtgcggaaacaaagccagcacctgttcctatgacagcgaagaaggttagacttgtccgtagaaataaacaaccagttgaacaaaaaagccgtggtgctttttgtgataaagaattttatccctgtgaagagggacgatgtcaacctgtagaggctcagcaagagtcttgctacggaagattgtattctgtaaaagtaaacgatgattgcaacgtagaaatttgccagtccgttccagaatacgctactgtaggatctccttaccctattgaaatccttgctataggcaaaaaagattgtgttgatgttgtgattacacaacagctaccttgcgaagctgaattcgtaagcagtgatccagaaacaactcctacaagtgatgggaaattagtctggaaaatcgatcgcctgggtgcaggagataaatgcaaaattactgtatgggtaaaacctcttaaagaaggttgctgcttcacagctgctactgtatgtgcttgcccagagctccgttcttatactaaatgcggtcaaccagccatttgtattaagcaagaaggacctgactgtgcttgcctaagatgccctgtatgctacaaaatcgaagtagtgaacacaggatctgctattgcccgtaacgtaactgtagataatcctgttcccgatggctattctcatgcatctggtcaaagagttctctcttttaacttaggagacatgagacctggcgataaaaaggtatttacagttgagttctgccctcaaagaagaggtcaaatcactaacgttgctactgtaacttactgcggtggacacaaatgttctgcaaatgtaactacagttgttaatgagccttgtgtacaagtaaatatctctggtgctgattggtcttacgtatgtaaacctgtggagtactctatctcagtatcgaatcctggagacttggttcttcatgatgtcgtgatccaagatacactcccttctggtgttacagtactcgaagctcctggtggagagatctgctgtaataaagttgtttggcgtattaaagaaatgtgcccaggagaaaccctccagtttaaacttgtagtgaaagctcaagttcctggaagattcacaaatcaagttgcagtaactagtgagtctaactgcggaacatgtacatcttgcgcagaaacaacaacacattggaaaggtcttgcagctacccatatgtgcgtattagacacaaatgatcctatctgtgtaggagaaaatactgtctatcgtatctgtgtaactaaccgtggttctgctgaagatactaacgtatctttaatcttgaagttctcaaaagaacttcagccaatagcttcttcaggtccaactaaaggaacgatttcaggtaataccgttgttttcgacgctttacctaaactcggttctaaggaatctgtagagttttctgttaccttgaaaggtattgctcccggagatgctcgcggcgaagctattctttcttctgatacactgacttcaccagtatcagacacagaaaatacccacgtgtat

SEQ ID NO: 30 - protein, 556 aa, Chlamydia pneumoniae [sequence omitted]

SEQ ID NO: 31 - DNA, 1452 bp, Chlamydia pneumoniae
  CDS (1)..(1452)
atgatcacacgcactaaaattatttgcactatagggccagcaacgaatagtccagagatgttagcaaaacttctagatgctgggatgaacgtagcaagattaaatttcagtcatgggagtcacgaaactcatggacaggctattggatttctcaaggagttaagggagcagaagcgggttcctttagcaattatgctagatactaaggggcctgaaattcgtttagggaatattcctcagccaatttcggtttctcagggacaaaagcttcgtctggtaagtagtgatatcgatgggagtgctgaagggggagtgtctctctatcctaaggggatatttccctttgttcctgagggtgctgatgttttaatagatgatggctacattcatgctgttgttgtctcttcagaggctgattctttagaattagagtttatgaacagtggccttctcaagtctcataaatctttgagtatccgaggtgttgatgttgctcttccctttatgacagagaaagatattgcggatcttaagtttggggtagagcagaatatggatgtggttgctgcatcttttgtgcgctacggtgaagatattgaaactatgcgcaagtgtttagcagacttaggcaatcctaagatgcccatcattgcaaaaatagaaaatcgtttaggggtagaaaatttctctaagattgccaagcttgcggatggaattatgattgctagaggagatttaggaatcgagctttctgtcgttgaagtcccaaatttgcaaaagatgatggctaaggtttctagagaaacaggtcacttctgtgtgactgcaacgcagatgctagaatctatgattcgcaatgtcttacctacacgagctgaagtctctgatattgccaatgcaatttatgatggttcttcagcagtgatgttgtcaggggaaactgcatctggagcccatcccgtggctgccgtgaaaatcatgcgttctgtgattttagaaacagaaaagaatctctcccatgattcattcttaaaattagacgatagcaatagcgctcttcaggtgtccccctatctctcagccattggattggcaggcattcagattgcagaaagggcagacgccaaagctcttattgtttatacagaatcaggaagttctccgatgtttctctctaaatatcgtccgaaattccctatcattgccgtgactccaagcacttctgtttactatcgcctagctttggaatggggggtctatcctatgcttacccaggaaagtgatcgcgctgtatggagacatcaggcctgtatttatggcatagaacagggcattctctctaattatgatcggattcttgtgcttagcagaggagcctgtatggaagaaacaaataatcttaccctgacaatagtgaatgatattttgactgggtcggaatttcctgaaacc

SEQ ID NO: 32 - protein, 484 aa, Chlamydia pneumoniae
MITRTKIICTIGPATNSPEMLAKLLDAGMNVARLNFSHGSHETHGQAIGFLKELREQKRVPLAIMLDTKGPEIRLGNIPQPISVSQGQKLRLVSSDIDGSAEGGVSLYPKGIFPFVPEGADVLIDDGYIHAVVVSSEADSLELEFMNSGLLKSHKSLSIRGVDVALPFMTEKDIADLKFGVEQNMDVVAASFVRYGEDIETMRKCLADLGNPKMPIIAKIENRLGVENFSKIAKLADGIMIARGDLGIELSVVEVPNLQKMMAKVSRETGHFCVTATQMLESMIRNVLPTRAEVSDIANAIYDGSSAVMLSGETASGAHPVAAVKIMRSVILETEKNLSHDSFLKLDDSNSALQVSPYLSAIGLAGIQIAERADAKALIVYTESGSSPMFLSKYRPKFPIIAVTPSTSVYYRLALEWGVYPMLTQESDRAVWRHQACIYGIEQGILSNYDRILVLSRGACMEETNNLTLTIVNDILTGSEFPET

SEQ ID NO: 33 - DNA, 1953 bp, Chlamydia pneumoniae
  CDS (1)..(1953)
atggcaacacccgctcaaaaatcccctacatttcaagatcctagttttgtaagagagctaggcagtaaccaccctgtcttttccccgctaacgcttgaggaaagaggggagatggcaatagctcgagtccagcagtgtggatggaatcatacaattgttaaggtaagtcttattattcttgctcttcttactattttagggggaggattactcgtaggattgctgccagcagttcctatgtttattggaacaggtctgattgctttgggagccgttatatttgctttggctttgattttatgtctttatgattctcagggccttcctgaggaactccctccggttcctgaaccacaacaaattcagattgaagatttaagaaacgagaccagagaagttcttgaagggactcttttagaggttctcttaaaggatagagacgctaaggaccctgcggtgccccaggtggttgtagactgtgaaaagcgtcttggaatgttggatcgtaagctgcgacgtgaagaggagattctgtatcgctcgacggcccatcttaaagacgaggaaaggtatgagttcttgctggagctcttggaaatgcgtagtctggttgccgatcggctagaatttaaccgtagaagttatgagcgatttgttcaaggaattatgacagttagatcagaggagggggaaaaagagatttctcgtctacaagatctaatcagtttgcagcagcagacggtgcaagatttaaggagtcggatcgatgacgagcagaagagatgctggacggctttacaacgtattaaccaatctcagaaggatatacaacgggctcatgatcgcgaggcttcgcagcgtgcctgtgagggcacagagatggattgtgcagaacgccagcaactggagaaggatttaaggagacagctgaaatctatgcaggagtggattgagatgaggggcacaatccatcaacaagagaaggcttggcgtaagcagaatgccaaattagaaagattacaagaggatctgagacttactgggattgcttttgacgaacaatctctgttctatcgcgaatataaagagaaatatctgagtcagaaactagatatgcaaaagattttacaggaagtcaacgcagagaaaagtgagaaggcttgcttagagagtctggtccatgactatgagaagcagctcgaacaaaaagatgctaatctgaagaaagcagcagctgtttgggaagaagaattagggaagcagcaacaggaagactacgaacaaacccaagaaattagacgtctgagtacattcattcttgagtaccaggacagtctgcgtgaggcagaaaaagttgagaaagatttccaagagctacaacaaaggtatagccgtcttcaagaggagaaacaggtaaaagaaaaaatcttagaagaaagtatgaatcattttgccgatctctttgagaaggctcaaaaggaaaacatggcctacaagaagaagttagcggatttagagggtgccgctgctcctactgagatcggtgaggacgatgactgggtactcacagattctgcttctctcagccagaagaagatccgcgaactcgtggaagagaatcaagaactcctgaaagcacttgcatttaaatctaacgaattgactcaactggttgccgatgctgtagaagctgaaaaagaaatcagcaagcttcgagaacacatagaagagcagaaagaaggattacgagctcttgataagatgcatgcacaagcgatcaaagattgcgaagctgctcagagaaaatgctgtgaccttgagagccttctctctcctgttcgagaagatgctggaatgagatttgagctagaggtcgagcttcaaagattgcaagaagaaaatgcacagcttagagcggaggttgaaagactagagcaagagcaatttcaagga

SEQ ID NO: 34 - protein, 651 aa, Chlamydia pneumoniae
MATPAQKSPTFQDPSFVRELGSNHPVFSPLTLEERGEMAIARVQQCGWNHTIVKVSLIILALLTILGGGLLVGLLPAVPMFIGTGLIALGAVIFALALILCLYDSQGLPEELPPVPEPQQIQIEDLRNETREVLEGTLLEVLLKDRDAKDPAVPQVVVDCEKRLGMLDRKLRREEEILYRSTAHLKDEERYEFLLELLEMRSLVADRLEFNRRSYERFVQGIMTVRSEEGEKEISRLQDLISLQQQTVQDLRSRIDDEQKRCWTALQRINQSQKDIQRAHDREASQRACEGTEMDCAERQQLEKDLRRQLKSMQEWIEMRGTIHQQEKAWRKQNAKLERLQEDLRLTGIAFDEQSLFYREYKEKYLSQKLDMQKILQEVNAEKSEKACLESLVHDYEKQLEQKDANLKKAAAVWEEELGKQQQEDYEQTQEIRRLSTFILEYQDSLREAEKVEKDFQELQQRYSRLQEEKQVKEKILEESMNHFADLFEKAQKENMAYKKKLADLEGAAAPTEIGEDDDWVLTDSASLSQKKIRELVEENQELLKALAFKSNELTQLVADAVEAEKEISKLREHIEEQKEGLRALDKMHAQAIKDCEAAQRKCCDLESLLSPVREDAGMRFELEVELQRLQEENAQLRAEVERLEQEQFQG

SEQ ID NO: 35 - DNA, 699 bp, Chlamydia pneumoniae
  CDS (1)..(699)
atgaatcgtagagacatggtaataacagctgtcgtagtgaatgctatattgcttgtggctcttttcgtcacatcaaagcgtattggcgtcaaggactatgacgagggattccgtaattttgcttctagcaaggttacacaagcagtagtttcagaagaaaaagtcatagaaaagcctgtagtcgcagaagtgcctagccgtcctatcgctaaagagactctagctgcacagtttattgaaagtaagccggttattgtaaccacaccacccgtgcctgttgttagcgaaaccccagaagtgcctactgtggcagttccgcctcagcctgttcgtgagacagtaaaagaggaacaagctccttatgctactgttgtagtgaaaaaaggagattttctcgaacgcattgcgagagcaaatcatactaccgttgcaaaattgatgcagatcaatgatcttaccaccacccaacttaaaattggtcaggtcatcaaagtccctacgtctcaagatgtcagcaacgaaaaaactcctcaaacacagaccgcaaaccctgaaaattattatatcgtccaagaaggggatagcccgtggacaatagcattgcgtaaccatattcgattggatgatttgctaaaaatgaatgatctcgatgaatataaagcccggcgccttaagcctggagatcagttgcgcatacgt

SEQ ID NO: 36 - protein, 233 aa, Chlamydia pneumoniae
MNRRDMVITAVVVNAILLVALFVTSKRIGVKDYDEGFRNFASSKVTQAVVSEEKVIEKPVVAEVPSRPIAKETLAAQFIESKPVIVTTPPVPVVSETPEVPTVAVPPQPVRETVKEEQAPYATVVVKKGDFLERIARANHTTVAKLMQINDLTTTQLKIGQVIKVPTSQDVSNEKTPQTQTANPENYYIVQEGDSPWTIALRNHIRLDDLLKMNDLDEYKARRLKPGDQLRIR

SEQ ID NO: 37 - protein, 18 aa, Chlamydia pneumoniae
MATPAQKSCRLEQEQFQG

SEQ ID NO: 38 - protein, 16 aa, Chlamydia pneumoniae
TVQDLRSRIDDEQKRC

SEQ ID NO: 39 - protein, 16 aa, Chlamydia pneumoniae
MNRRDMVCPGDQLRIR

SEQ ID NO: 40 - protein, 16 aa, Chlamydia pneumoniae
VTSKRIGVKDYDEGFC

SEQ ID NO: 41 - DNA, 20 bp, Synthetic
ggatttattgggcgtaaagg

SEQ ID NO: 42 - DNA, 20 bp, synthetic
tccacatcaagtatgcatcg

SEQ ID NO: 43 - DNA, 20 bp, synthetic
gctgcaaactatactactgc

SEQ ID NO: 44 - DNA, 20 bp, synthetic
gaaaacatcaaagcgatccc

SEQ ID NO: 45 - DNA, 20 bp, synthetic
gtgatgggaaattagtctgg

SEQ ID NO: 46 - DNA, 20 bp, synthetic
atcctgtgttcactacttcg

SEQ ID NO: 47 - DNA, 20 bp, synthetic
agcagaagtttactctgtcg

SEQ ID NO: 48 - DNA, 20 bp, synthetic
ctactgatggaaacctaagc

SEQ ID NO: 49 - DNA, 30 bp, synthetic
aagatatcaaggctactgatgaggaaaccg

SEQ ID NO: 50 - DNA, 28 bp, synthetic
ttgatatctagaacttgctgcagcggga

SEQ ID NO: 51 - DNA, 20 bp, synthetic
gactactgctataggtaagg

SEQ ID NO: 52 - DNA, 20 bp, synthetic
gagatgctaagtttcctagc

SEQ ID NO: 53 - DNA, 20 bp, synthetic
tctctttcgtccattgatcg

SEQ ID NO: 54 - DNA, 20 bp, synthetic
ctcaggattgttagagtacc

SEQ ID NO: 55 - DNA, 20 bp, synthetic
gtccagtgaaatcatggccg

SEQ ID NO: 56 - DNA, 20 bp, synthetic
cccatgttttcatgtttgtc

SEQ ID NO: 57 - DNA, 20 bp, synthetic
tcaggaaatcaagtcgttcc

SEQ ID NO: 58 - DNA, 20 bp, synthetic
agattcctgagaacgtaagc

SEQ ID NO: 59 - DNA, 20 bp, synthetic
tgttgttgtctcttcagagg

SEQ ID NO: 60 - DNA, 20 bp, synthetic
ctaccccaaacttaagatcc

SEQ ID NO: 61 - DNA, 20 bp, synthetic
tcaatgatcttaccaccacc

SEQ ID NO: 62 - DNA, 20 bp, synthetic
gttacgcaatgctattgtcc

SEQ ID NO: 63 - DNA, 20 bp, synthetic
tgcatcttatcaagagctcg

SEQ ID NO: 64 - DNA, 20 bp, synthetic
gaagttagcggatttagagg

SEQ ID NO: 65 - DNA, 20 bp, synthetic
gaggagaactgataagaacg

SEQ ID NO: 66 - DNA, 20 bp, synthetic
cttaactcctgatctcatcc